(19) United States
(12) Patent Application Publication
(10) Pub. No.: US 2017/0275681 A1
(43) Pub. Date: Sep. 28, 2017

(54) SINGLE MOLECULE RNA DETECTION

(71) Applicant: The Regents of the University of California, Oakland, CA (US)

(72) Inventors: Sheng Zhong, San Diego, CA (US); Fernando Biase, San Diego, CA (US)

(21) Appl. No.: 15/462,646

(22) Filed: Mar. 17, 2017

Related U.S. Application Data
(63) Continuation of application No. PCT/US2015/051074, filed on Sep. 18, 2015.
(60) Provisional application No. 62/053,595, filed on Sep. 22, 2014.

(51) Int. Cl. C12Q 1/68

(52) U.S. Cl. CPC C12Q 1/6841; C12Q 1/6825

(57) ABSTRACT

Quantification of RNAs is one of the most essential tools to characterize cells. This tool is widely used in disease diagnosis, pharmacogenomics, and drug development. Single cell RNA fluorescent in situ hybridization (smRNA-FISH) revolutionized RNA detection and quantification by detecting every single RNA molecule of a gene. However, this technology is incapable of assaying relatively short RNAs, and it suffers from high cost and low throughput. Here, we describe a technology that simultaneously overcomes the three drawbacks using conventional instrumentation. This QD-smRNA-FISH technology uses hybridization of quantum dot-labeled DNA oligonucleotides to the RNA molecules for visualization and counting. Quantum dots (QDs) have been assumed inapplicable to counting individual RNA molecules, due to the well-known blinking problem (display intermittency) (Medintz I L, Uyeda H T, Goldman E R, Mattoussi H (2005) Quantum dot bioconjugates for imaging, labelling and sensing. Nat Mater 4: 435-446). This problem has been circumvented by this new experimental design. In some embodiments described herein, the methods assemble several QDs to every target RNA molecule and leverage the complementation of the QDs to achieve an overall non-intermittent signal on each target molecule. We validated QD-smRNA-FISH by comparing its signals with those of standard smRNA-FISH. We successfully applied QD-smRNA-FISH to test the interaction of two RNAs, a task that cannot be accomplished with standard smRNA-FISH. The QD-smRNA-FISH method offers a highly accurate method for single RNA molecule detection and counting under standard fluorescent microscopes, and enables analysis of relatively short RNAs (<1000 bases) which comprises the majority of eukaryotic transcriptome and more than half of the eukaryotic mRNAs. The QD-smRNA-FISH method also reduces the reagent cost by several folds and allows for analysis of multiple genes in parallel.

### CROSS REFERENCE TO RELATED APPLICATIONS

The present application is a continuation of PCT/US2015/051074 filed Sep. 18, 2015, which claims the benefit of priority to U.S. Provisional Patent Application No. 62/053,595, entitled SINGLE MOLECULE RNA DETECTION, filed on Sep. 22, 2014. The entire disclosures of the aforementioned applications are expressly incorporated herein by reference in their entirety.

### STATEMENT REGARDING FEDERALLY SPONSORED R&D

This invention was made with government support under grant number NIH DP2-OD007417 awarded by the National Institute of Health. The government has certain rights in the invention.

### REFERENCE TO SEQUENCE LISTING, TABLE, OR COMPUTER PROGRAM LISTING

The present application is being filed along with a Sequence Listing in electronic format. The Sequence Listing is provided as a file entitled UCSD090-001WO_Sequence_Listing.TXT, created Sep. 11, 2015, which is 26 Kb in size. The information in the electronic format of the Sequence Listing is incorporated herein by reference in its entirety.

### BACKGROUND OF THE INVENTION

### Field of the Invention

Methods and compositions for detecting at least one target nucleic acid are provided herein.

### Description of the Related Art

Current methods of detecting target nucleic acids may require the use of a large number of probes or may be expensive. Accordingly, improved methods and compositions for use therein are beneficial.

### SUMMARY OF THE INVENTION

Some embodiments of the present invention are provided in the following numbered paragraphs:

1. A method for detecting at least one target nucleic acid comprising contacting said at least one target nucleic acid with a plurality of different nucleic acid probes which are associated with at least one detectable component with a high wavelength emission under conditions in which said plurality of different nucleic acid probes bind to said at least one target nucleic acid.

2. The method of Paragraph 1, wherein said at least one detectable component comprises a particle with a high wavelength emission.

3. The method of Paragraph 2, wherein said particle is a quantum dot.

4. The method of any one of Paragraphs 1-3, wherein said target nucleic acid comprises RNA.

5. The method of any one of Paragraphs 1-5, wherein said target nucleic acid comprises a single RNA molecule within a single cell.

6. The method of any one of Paragraphs 1-5, wherein said plurality of different probes comprise 5 or more different probes.

7. The method of any one of Paragraphs 1-6, wherein each of said plurality of different probes is between about 10 and about 100 nucleotides in length.

8. The method of any one of Paragraphs 1-7, wherein each of said plurality of different probes is between about 20 and about 80 nucleotides in length.

9. The method of any one of Paragraphs 1-8, wherein each of said plurality of different probes is about 30 nucleotides in length.

10. The method of any one of Paragraphs 1-9, wherein a plurality of target nucleic acids are detected.

11. A nucleic acid probe associated with at least one detectable component with a high wavelength emission.

12. The nucleic acid probe of Paragraph 11, wherein said at least one detectable component comprises a particle with a high wavelength emission.

13. The nucleic acid probe of Paragraph 12, wherein said particle is a quantum dot.

14. The nucleic acid probe of any one of Paragraphs 11-13, wherein said at least one detectable component is covalently linked to said nucleic acid probe.

15. The nucleic acid probe of any one of Paragraphs 11-14, wherein said nucleic acid probe is between about 10 and about 100 nucleotides in length.

16. The nucleic acid probe of any one of Paragraphs 11-15, wherein said nucleic acid probe is between about 20 and about 80 nucleotides in length.

17. The nucleic acid probe of any one of Paragraphs 11-16, wherein said nucleic acid probe is about 30 nucleotides in length.

18. A kit comprising a plurality of different nucleic acid probes which are able to hybridize to at least one target nucleic acid, wherein each of said plurality of different nucleic acid probes is associated with at least one detectable component with a high wavelength emission.

19. The kit of Paragraph 18, wherein said at least one detectable component comprises a particle with a high wavelength emission.

20. The kit of any one of Paragraph19, wherein said particle is a quantum dot.

21. The kit of any one of Paragraphs 18-20, wherein said at least one detectable component is covalently linked to each of said plurality of different nucleic acid probes.

22. A method for detecting a plurality of target nucleic acids is provided, wherein the method comprises contacting said plurality of target nucleic acids with a plurality of sets of nucleic acid probes, wherein each set of nucleic acid probes comprises a plurality of different nucleic acid probes which are associated with at least one detectable component with a high wavelength emission, wherein each set of nucleic acid probes hybridizes to a different target nucleic acid, and wherein each set of nucleic acid probes is associated with a detectable component which emits at a high wavelength which is distinguishable from the high wavelength emissions of the detectable components associated with the other sets of nucleic acid probes and wherein said contacting is performed under conditions in which said plurality of sets of nucleic acid probes bind to said plurality of target nucleic acids.

### BRIEF DESCRIPTION OF THE DRAWINGS

FIG. 1. Schematics of the coupling of oligonucleotides and streptavidin coated quantum dots. As shown, the sequences that are light in color are complimentary to the oligonucleotide probes that are coupled with quantum dots.

FIG. 2A to 2D. Steps of spot identification and counting of single molecule ribonucleic acid. (FIG. 2A) Original raw image (slice number 29 of 71 z-stacks) acquired with 60× oil objective. Scale bar represents approximately 1 μm. (FIG. 2B) Image after Laplacian of Gaussian filter is applied. (FIG. 2C) Deconvolved image and converted to 16 bit format. (FIG. 2D) Digital identification of signal by intensity threshold specification.

FIG. 3A to 3B. Digital 3D reconstruction of the spots detected and counted. (FIG. 3A) Lateral and (FIG. 3B) top perspectives for visualization of the spots counted.

FIG. 4A to 4D. Quantification of RNA molecules with QD-smRNA-FISH. (FIG. 4A) Scheme of single molecule RNA-FISH with probes labeled with quantum dots. As shown the sequences that are light in color are complimentary to the probes (FIG. 4B) Quantification Actb RNA molecules in stem cells. As shown replicate 1 is at position 13, 14, 15, 19, 22, 23, 27, 30-33, and 37. (FIG. 4C) Position of probes for smRNA-FISH, or QD-smRNA-FISH relative to Actb gene. (FIG. 4D) Co-localization of signals detected (arrows) from probes labeled with organic (Alexa555) and inorganic (QD 565) dyes.

FIG. 5A to 5E. Experimental evidence of co-localization of RNAs from 5Malat1 and Slc2a3 genes. (FIG. 5A) Depiction of dual labeling experiment to test the hypothesis of co-localization of Malat1 and Slc2a3 RNAs. Quantification of (FIG. 5B) Malat1 and (FIG. 5C) Slc2a3 RNA molecules. (FIG. 5D) Quantification of co-localized RNA molecules of Malat1 and Slc2a3. As shown the bottom of the graph represents Malat1, the regions with the corresponding numbers is the overlap, and the top parts of the graph represent Slc2a3. (FIG. 5E) Representative images obtained with different dyes and filters showing the co-localization of four spots (arrows).

FIG. 6. Titration of quantum dots and oligonucleotides for testing the optimal mixture for probe labeling. The qDot625+ Actb oligos were labeled with a red label while the Actb oligos were labeled with a green label. Regions on the gel where the qDot625+ Actb oligos and Actb oligos migrate are indicated on the gel.

FIG. 7A to 7B. Count of spots at progressive fluorescence thresholds. Inset images are the counts around the first plateau of three consecutive counts. As shown the region comprising arbitrary fluorescence units of 17000 to 27000 are shown in the inset graph (left panel, FIG. 7A), and the region comprising arbitrary fluorescence units of 30000 to 40000 are shown in the inset graph (right, FIG. 7B).

FIG. 8A to 8C. Distinctions of Alexa 555 and qDot 565 excitation and emission. (FIGS. 8A and 8B) The excitation wave lengths of qDots and Alexa 555 were distinct (Exciter lane, b). (FIG. 8C) RNA-FISH signals of Alexa 555 and qDot 565 acquired with corresponding and exchanged emission filters.

FIG. 9A to 9C. Distinction of qDot 525 and qDot 605 signals. The emission wave lengths (solid lines) of qDot 525 and qDot 605 were separated The left peak is Qdot 525 and the right peat is Qdot 605. (FIG. 9A), coupled with emission filters of non-overlapping ranges (Emitter lane, FIG. 9B) (images drawn with Fluorescence SpectraViewer, Life Technologies). (FIG. 9C) RNA-FISH signals of qDot 525 and qDot 605 acquired with corresponding and exchanged emission filters.

FIG. 10A to 10D. Calling co-localized RNAs with two-color smRNA-FISH. (FIG. 10A) Voxel distribution of all spots detected (each corresponding to one RNA molecule). Center dot: average size. Error bar: sample standard deviation. (FIG. 10B) A depiction of the average volume and radius of identified spots. (FIG. 10C) The criterion for calling co-localized spots. (FIG. 10D) Graphical representation of all the spots identified in a field of view covering 2 cells. Arrows point to co-localized spots imaged with dyes emitting fluorescence at different wavelengths. 1 pixel˜107 nm. The arrows represent co-localization between Malat1 and Scl2a3.

FIG. 11. As shown is the fluorescence spectra of CdTe quantum dots of various sizes. The different sized quantum dots emit different color light due to the quantum confinement.

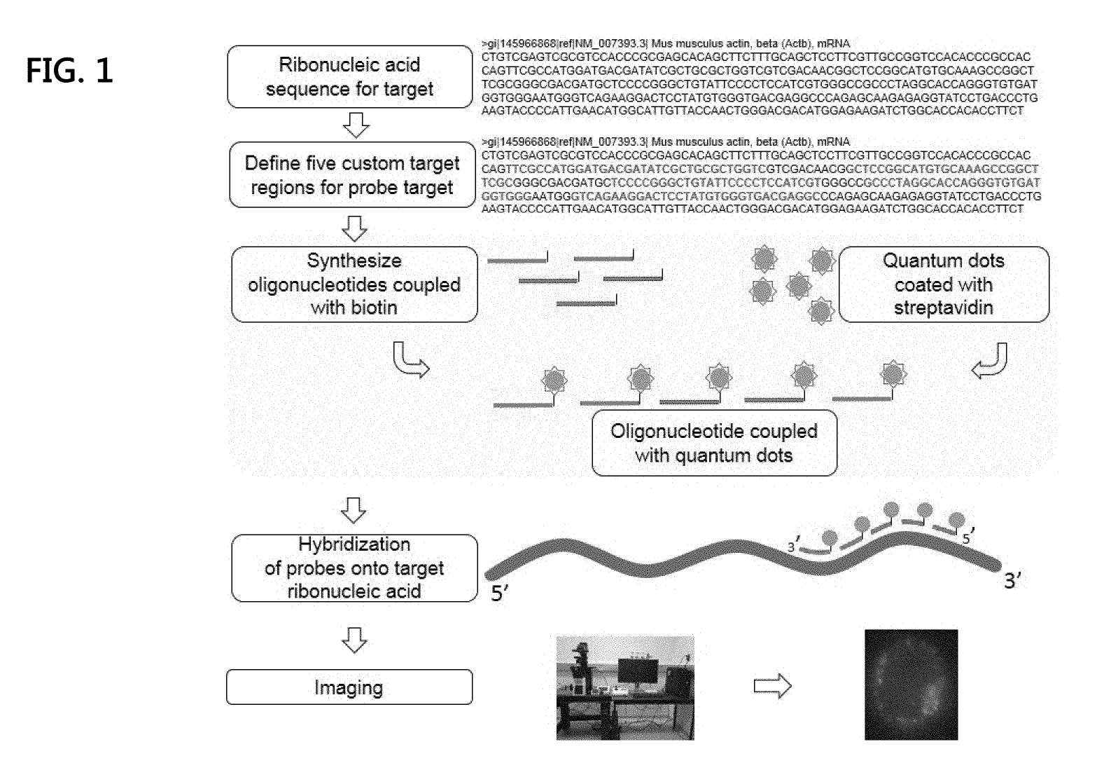

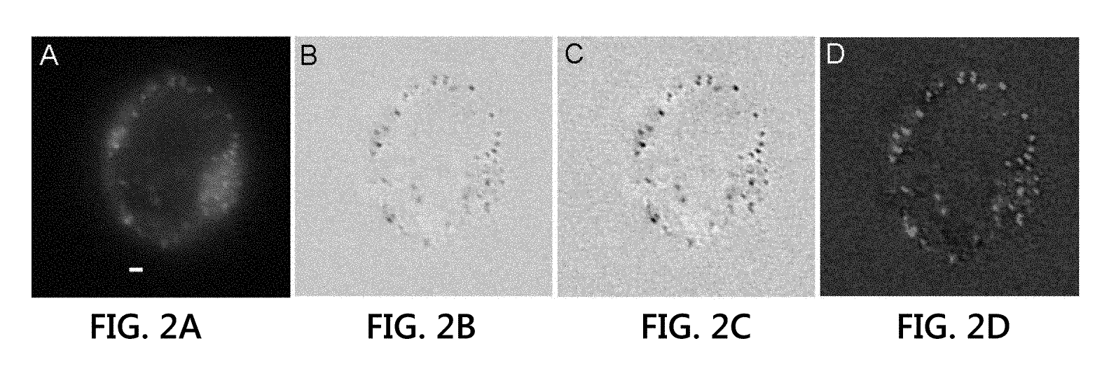

### DEFINITIONS

In the description that follows, a number of terms are used extensively.

The following definitions are provided to facilitate understanding of the present alternatives.

As used herein, “a” or “an” may mean one or more than one.

As used herein, the term “about” indicates that a value includes the inherent variation of error for the method being employed to determine a value, or the variation that exists among experiments.

As used herein, a “nucleic acid probe” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action, that can be used to detect the presence of a target nucleic acid (i.e. a DNA target or an RNA target). Nucleic acid probes can be composed of monomers that are naturally-occurring nucleotides (such as DNA and RNA), or analogs of naturally-occurring nucleotides (e.g., enantiomeric forms of naturally-occurring nucleotides), or a combination of both. The nucleic acid probe or hybridization probe when labeled with quantum dots or comparable particles that emit at a high wavelengths, can be used to identify complementary segments or sequences present in the nucleic-acid sequences of various microorganisms. The nucleic acid probe can comprise a fragment of DNA or RNA of variable length. The size of the probe can range in size from about 10, about 20, about 30, about 40, about 50, about 60, about 70, about 80, about 90, about 100, about 200, about 300, about 400, about 500, about 600, about 700, about 800, about 900 or about 1000 bases long or any other length in between any two of the aforementioned values. The target nucleic acid sequence can comprise a size of about 10, about 20, about 30, about 40, about 50, about 60, about 70, about 80, about 90, about 100, about 200, about 300, about 400, about 500, about 600, about 700, about 800, about 900 or about 1000 bases long or any other length in between any two of the aforementioned values. It can then be used in DNA or RNA samples to detect the presence of nucleotide sequences (the DNA target or RNA target strand) that are complementary to the sequence in the probe. The probe thereby hybridizes to single-stranded nucleic acid (DNA or RNA) whose base sequence allows probe-target base pairing due to complementarity between the probe and target. The probe can also be first denatured (for example, by heating or under alkaline conditions such as exposure to sodium hydroxide) into single stranded DNA (ssDNA) and then hybridized to the target ssDNA or RNA. In some embodiments, a method for detecting at least one target nucleic acid is provided, wherein the method comprises contacting said at least one target nucleic acid with a plurality of different nucleic acid probes which are associated with at least one detectable component with a high wavelength emission under conditions in which said plurality of different nucleic acid probes bind to said at least one target nucleic acid. In some embodiments, the nucleic acid is a DNA. In some embodiments, the nucleic acid is an RNA. In some embodiments, the probe binds to a target sequence within the target nucleic acid strand.

“Target” or “Target nucleic acid” as referred to herein, is a nucleic acid sequence that is complementary to the nucleic acid probe. In some embodiments, the target nucleic acid comprises a single RNA molecule within a single cell. In some embodiments, the target nucleic acid is within a cell or is in a nucleic acid sample obtained from a cell or a plurality of cells. In some embodiments, the nucleic acid probes are complimentary to a target sequence on a single target nucleic acid strand. In some embodiments, the target nucleic acid strand is an RNA. In some embodiments, the target nucleic acid strand comprises at least one target sequence. In some embodiments at least one nucleic acid probe is complimentary to at least one target sequence on a target nucleic acid strand.

“RNA” refers to ribonucleic acid and is a polymeric molecule implicated in various biological roles in coding, decoding, regulation, and expression of genes. RNA plays an active role within cells by catalyzing biological reactions, controlling gene expression, or sensing and communicating responses to cellular signals. Messenger RNA carries the information of proteins sequences to a ribosome, through which it is translated. In some embodiments, a method for detecting at least one target nucleic acid is provided, wherein the method comprises contacting said at least one target nucleic acid with a plurality of different nucleic acid probes which are associated with at least one detectable component with a high wavelength emission under conditions in which said plurality of different nucleic acid probes bind to said at least one target nucleic acid. In some embodiments, the target nucleic acid comprises a single RNA molecule within a single cell. In some embodiments, the method further comprises single molecule analysis of RNA splicing and RNA-RNA interaction in vivo.

The term “complementary to” means that the complementary sequence is complementary to all or a portion of a reference polynucleotide sequence. For illustration, the nucleotide sequence 5′-“CATTAG”-3′ corresponds to a reference sequence “CATTAG” and is complementary to a reference sequence 3′-“GTAATC”-5′. In some embodiments, a method for detecting at least one target nucleic acid is provided, wherein the method comprises contacting said at least one target nucleic acid with a plurality of different nucleic acid probes which are associated with at least one detectable component with a high wavelength emission under conditions in which said plurality of different nucleic acid probes bind to said at least one target nucleic acid. In some embodiments, the different nucleic acid probes are complimentary to at least one target nucleic acid. In some embodiments, the at least one target nucleic acid is a DNA or an RNA. In some embodiments, the target nucleic acid is a messenger RNA (mRNA).

“Particle” as described herein, refers to a minute object, such as a nanocrystal, for example, which can undergo a particle decay from a high energy state to a lower energy state by emitting with a high wavelength emission. In some embodiments described herein, the particle emits at a high emission wavelength of about 450 nm, about 500 nm, about 550 nm, about 600 nm, about 650 nm, about 700 nm, about 750 nm, about 800 nm or about 850 nm, or any other high emission wavelength between any two aforementioned values.

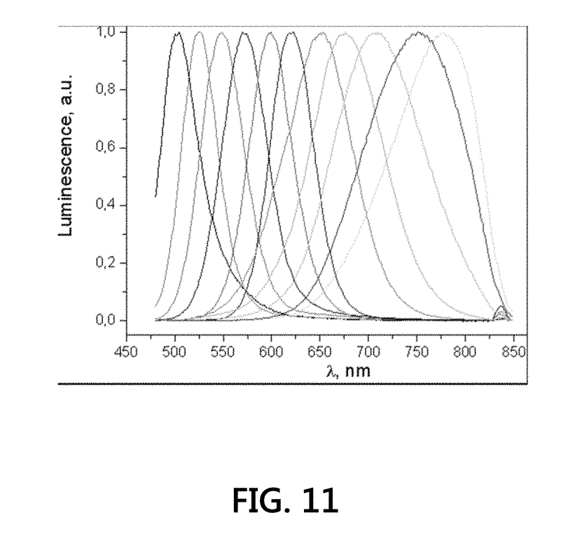

“Quantum dot” as described, refers to an inorganic nanocrystal semiconductor. The size of the quantum dot reflects the wavelength of light emitted which allows for a highly tunable color spectrum. The size of the quantum dot is controllable and an increase in size can produce an increased wavelength range of emission. As such, the quantum dots display unique electronic properties that are the result of the high surface to volume ratios for the particles. The most apparent result is their fluorescence, in which they can produce a distinct color that is determined by their size. Quantum dots can range in size from about 1, about 2, about 3, about 4, about 5, about 6, about 7, about 9, about 10 or about 11 nanometers. In some embodiments described herein, the quantum dot comprises a size from about 1, about 2, about 3, about 4, about 5, about 6, about 7, about 8, about 9, about 10 or about 11 nanometers or any other size in between any two aforementioned values. As shown in FIG. 11, the exemplified quantum dots can emit within a very narrow range of wavelengths, but excited across a wide spectrum which allows multiplexing or combining different nucleic acid probes with different sized quantum dots which emit at different wavelengths. As shown in FIG. 11, the exemplary quantum dots can emit between 450 and 850 nm. The larger the size of the quantum dot, the redder its fluorescence spectrum (lower energy). Conversely, small dots emit bluer light (higher energy). Without being limiting, in Table 1 below, are exemplary sizes that can correlate with the emission peak and color emitted.

TABLE 1
Quantum dot size correlation with emission wavelength.

| Size (nm) | Emission Peak (nm) | Color |
|---|---|---|
| 2.2 | 495 | blue |
| 2.9 | 550 | green |
| 3.1 | 576 | yellow |
| 4.1 | 595 | orange |
| 4.4 | 611 | orange |
| 4.8 | 644 | red |
| 7.3 | 655 | dark red |

The emission of the quantum dots are seen at high wavelengths, such as wavelengths between 450 and 850 nm. In some embodiments the quantum dot emits at a high emission wavelength of about 450 nm, about 500 nm, about 550 nm, about 600 nm, about 650 nm, about 700 nm, about 750 nm, about 800 nm or about 850 nm, or any other high emission wavelength between any two aforementioned values. In some embodiments described herein, the quantum dot comprises a size from about 1, about 2, about 3, about 4, about 5, about 6, about 7, about 8, about 9, about 10 or about 11 nanometers or any other size in between any two aforementioned values.

Quantum dots can be made by a variety of binary compounds. Without being limiting, the quantum dots can be made of, for example, lead sulfide, lead selenide, cadmium selenide, cadmium sulfide, indium arsenide, and indium phosphide. In some embodiments described herein, the quantum dots comprise lead sulfide, lead selenide, cadmium selenide, cadmium sulfide, indium arsenide, or indium phosphide.

To detect hybridization of the probe to its target sequence, the probe can also be tagged (or “labeled”) with a quantum dot or a comparable particle that emits at a high wavelength. DNA sequences or RNA transcripts that have moderate to high complementarity to the probe are then detected by visualizing the hybridized probe via imaging techniques known to one skilled in the art. Detection of sequences with moderate or high similarity depends on how stringent the hybridization conditions were applied such as, for example, high stringency, such as high hybridization temperature and low salt in hybridization buffers, permits only hybridization between nucleic acid sequences that are highly similar, whereas low stringency, such as lower temperature and high salt, allows hybridization when the sequences are less similar.

Depending on the method, the probe may be synthesized using the phosphoramidite method, or it can be generated and labeled by PCR amplification or cloning (both are older methods). Instances wherein the probe is utilized in vivo, in order to increase the in vivo stability of the probe, RNA is not preferably used, instead RNA analogues may be used, in particular morpholino-derivatives. Molecular DNA- or RNA-based probes can be used in screening gene libraries, detecting nucleotide sequences with blotting methods, and in other gene technologies, such as nucleic acid and tissue microarrays, for example. In some embodiments, the probe is synthesized by a phosphoramidite method, or generated and labeled by PCR amplification or cloning. In some embodiments, the probe comprises RNA analogues such as, for example, morpholino-derivatives.

“Emission spectrum” as defined herein refers to a spectrum of frequencies of electromagnetic radiation that is emitted due to an atom or a molecule making a transition from a high energy state to a lower energy state.

### DETAILED DESCRIPTION OF THE PREFERRED EMBODIMENT

Disclosed is a method for detecting at least one target nucleic acid. In some embodiments, the at least one target nucleic acid is a single RNA molecule within a single cell. In some embodiments, the method involves using quantum dots. In some embodiments, the method reduces cost by about ⅓.

One embodiment relates to a method comprising use of two or more (in some embodiments at least five) nucleic acid probes capable of binding to an RNA (or other nucleotide) target. In some embodiments, the nucleic acid probes are about 30 nucleotides in length. In some embodiments, the nucleic acid probes are between about 10 to about 100 nucleotides in length. In some embodiments, the nucleic acid probe is about 5, about 10, about 20, about 30, about 40, about 50, about 60, about 70, about 80, about 90, about 100, or about 110 nucleotides in length, or any number of nucleotides in between any two aforementioned values. In some embodiments, the nucleic acid probes are between about 20 about 80 nucleotides in length. In some embodiments described herein, the nucleic acid probes are between about 20, about 30, about 40, about 50, about 60, about 70 or about 80 nucleotides in length, or any length in between any two aforementioned values. In some embodiments, each nucleic acid probe comprises at least one quantum dot (or a comparable particle characterized by a high wavelength emission). In some embodiments, the quantum dot comprises a size from about 1, about 2, about 3, about 4, about 5, about 6, about 7, about 8, about 9, about 10 or about 11 nanometers or any other size in between any two aforementioned values. In some embodiments, the at least one quantum dot or comparable particle characterized by a high wavelength emission emits at a high emission wavelength of about 450 nm, about 500 nm, about 550 nm, about 600 nm, about 650 nm, about 700 nm, about 750 nm, about 800 nm or about 850 nm, or any other high emission wavelength between any two aforementioned values.

In some embodiments, the method may be used to detect one or more RNA target. In some embodiments, the method further comprises single molecule analysis of RNA splicing and RNA-RNA interaction in vivo.

The current methods rely on use of about 40 nucleic acid probes comprising about 20 nt, linked to fluorescent dyes. This large number of probes is required because current dyes have limited emission spectrums. This results in a cost of about $1,500/RNA target. Use of quantum dots (QD) have not been previously considered a solution to this problem due to their inherent ‘blinking’. Thus, in use, there is about a 10% chance that when viewing a sample, the QD may not be emitting. However, in some embodiments, the abovementioned concerns are avoided by using a plurality of QD-labeled probes (preferably about five) to a same target. This allows for five QD being linked to a given RNA target, thus the chance of all QD not emitting simultaneously is about 105. This reduces a number of probes from about 40 to about 5. Also, there may be increased probe-target specificity and signal-to-noise ratio.

In some embodiments, the method permits assessment of a plurality of RNA targets simultaneously. This is beneficial since, currently assessing a plurality of RNA targets simultaneously is difficult and requires use of expensive microscopes. In some embodiments, the method allows use of less expensive microscopes commonly used in cell imaging labs. In some embodiments, the method may be used to detect one or more RNA target. In some embodiments, the method further comprises single molecule analysis of RNA splicing and RNA-RNA interaction in vivo.

Some applications of the present method include detection of single RNA molecules in a cell. In some embodiments, the method may be used with microarray technologies.

In some embodiments, the method may detect single RNA molecules in cells and thus digitally quantify the RNA transcripts of any gene. In some embodiments, the method may be used to detect one or more RNA target. In some embodiments, the method further comprises single molecule analysis of RNA splicing and RNA-RNA interaction in vivo. In some embodiments, the cell is a eukaryotic or a prokaryotic cell. In some embodiments, wherein the cell is a eukaryotic cell, the cell is a human cell, a cancer cell, a macrophage, a lymphocyte, a tumor cell, a precancerous cell or a microglial cell. Depending upon the analysis, which can be used to track gene expression by the presence of RNA or DNA in a cell, the cell type can be used for the detection in order to analyze a disease, the progression of a disease or to predict a specific disease by the presence or absence of a number of target nucleic acids within the said cell. In some embodiments, the method uses hybridization of quantum dot-labeled single stranded DNA oligonucleotides to the RNA targets for microscopic visualization and quantification. Previously quantum dots were thought not to be capable of single molecule studies due a well-known blinking problem. In some embodiments, this problem may be reduced or overcome. In some embodiments, the method reduces costs relative to technologies that use organic dyes and many fold more oligonucleotides for single molecule RNA-FISH to less than ⅓ of the cost of such technologies, while offering clearer, easier to detect, and more reliable signals. In some embodiments, the quantum dots comprise a size from about 1, about 2, about 3, about 4, about 5, about 6, about 7, about 8, about 9, about 10 or about 11 nanometers or any other size in between any two aforementioned values. In some embodiments, the quantum dot emits at a high emission wavelength of about 450 nm, about 500 nm, about 550 nm, about 600 nm, about 650 nm, about 700 nm, about 750 nm, about 800 nm or about 850 nm, or any other high emission wavelength between any two aforementioned values.

The majority of the scientific discoveries of a given gene are obtained by studying its transcription. The information obtained from this biological phenomenon (gene transcription) is used to infer important parameters related to gene functions, from the structure of the coding sequence, all the way to the potential interactions at the protein level. While the majority of the information of gene expression has been collected from cell populations (for example: cell culture, or tissue samples), an expansive area or research has directed the attention for the individual cells. As a consequence, the importance of a single cell for the composition a tissue has gained attention from researchers and funding sources. In some embodiments, the method further comprises obtaining transcription levels of a gene of interest. In some embodiments, obtaining of transcription levels of a gene of interest can be used to analyze a disease, the progression of a disease or to predict a specific disease by the presence or absence of a number of target nucleic acid within the said cell.

Although very important, studying the gene expression of a single cell brings along technical challenges. Currently, only one technique is capable of obtaining information of gene expression of a single cell without the introduction of technical biases (Raj, A., Peskin, C. S., Tranchina, D., Vargas, D. Y., and Tyagi, S. (2006). Stochastic mRNA synthesis in mammalian cells. PLoS Biol. 4, e309). This technique is the fluorescent in situ hybridization, and a recent technological improvement has allowed researchers to image a single molecule of ribonucleic acid of a gene of interest using a microscope (Raj, A., van den Bogaard, P., Rifkin, S. A., van Oudenaarden, A., and Tyagi, S. (2008). Imaging individual mRNA molecules using multiple singly labeled probes. Nat. Methods 5, 877-879; Batish, M., Raj, A., and Tyagi, S. (2011). Single molecule imaging of RNA in situ. Methods Mol. Biol. 714, 3-13. Raj, A., and Tyagi, S. (2010). Detection of individual endogenous RNA transcripts in situ using multiple singly labeled probes. Methods Enzymol. 472, 365-386; Shaffer, S. M., Wu, M.-T., Levesque, M. J., and Raj, A. (2013). Turbo FISH: A Method for Rapid Single Molecule RNA FISH. PLoS One 8, e75120). Even though this advancement was a great step towards the study of gene expression, in some embodiments, the present methods and compositions greatly improve the targeted detection of single molecules of ribonucleic acid at a much lower cost compared to the current methods.

In some embodiments, methods to detect single molecules of nucleic acids by fluorescent in situ hybridization using a plurality of different nucleic acid probes coupled with quantum dots are provided. For example, in some embodiments, five different nucleic acid probes associated with quantum dots are utilized. In some embodiments, the quantum dots can be covalently linked to the nucleic acid probes. In some embodiments, the nucleic acid probes associated with quantum dots can be used to detect single molecules. The oligonucleotides associated with quantum dots may also be used to count the number of RNAs (in cultured cells or tissues) with a new technique. In some embodiments, the methods bring more than one (a designed number) of quantum dots to a target molecule (such as RNA). For example, in some embodiments, multiple quantum dots are brought to the proximity of the target molecule complement each other's signal, thus “cancelling out” the lost signal when “blinking”. The loss of signal of quantum dots, dubbed blinking, has been a barrier to applying them for detecting and quantifying molecules.

The quantum dots can be conjugated to the nucleic acid probes by a variety of techniques that are known to a person skilled in the art. Without being limiting, quantum dots can be conjugated to a nucleic acid probe in the presence of EDC an N-hydroxysuccinimide (NHS) (Choi et al. In situ visualization of gene expression using polymer-coated quantum-dot-DNA conjugates. Small 2009; 5:2085e91; incorporated by reference in its entirety), amine modification of DNA for coupling to quantum dots surfaces via the formation of an amide linkage, and biotinylation of oligonucleotides for attachment to quantum dots coated in streptavidin (Kim et al., Conjugation of DNA to Streptavidin-coated Quantum Dots for the Real-time Imaging of Gene Transfer into Live Cells, NSTI-Nanotech 2004, www.nsti.org, ISBN 0-9728422-9-2 Vol. 3, 2004; incorporated by reference in its entirety. In some embodiments, the quantum dot is conjugated to the nucleic acid in the presence of EDC an N-hydroxysuccinimide (NETS). In some embodiments, the quantum dot is conjugated to the nucleic acid by amine modification of the nucleic acid for coupling to quantum dots surfaces via the formation of an amide linkage. In some embodiments, the quantum dot is conjugated to the nucleic acid by biotinylation of the nucleic acid for attachment to quantum dots coated in streptavidin. In some embodiments, wherein the nucleic acid probe is complimentary to the target nucleic acid sequence, the probe further comprises a linker nucleic acid to which the QD is covalently attached. In some embodiments, the linker DNA allows freedom in the motion of the quantum dots during annealing and furthermore, can allow the quantum dot a distance from the annealing site in the event that the size of the QD can prevent annealing. Methods to improve annealing conditions can further be performed and are known to one skilled in the art. For example, a short linker which is not complementary to the target binding site can comprise about 3, about 4, about 5, about 6, about 7, about 8, about 9 or about 10 nucleic acids or any number of nucleic acids in between any two of the aforementioned values. In some embodiments of the nucleic acid probe, the probe further comprises a nucleic acid linker, wherein the nucleic acid linker is not complementary to the target binding site, wherein the nucleic acid linker comprises about 3, about 4, about 5, about 6, about 7, about 8, about 9 or about 10 nucleic acids or any number of nucleic acids in between any two of the aforementioned values, and wherein the QD is covalently attached to the nucleic acid linker.

By using a small (pre-designed) number of different nucleic acid probes, designed to hybridize onto at least one specific sequence of nucleic acid, and which is coupled with quantum dots, allows the detection of single molecules of the targeted nucleic acid. In some embodiments, the cumulative fluorescence of a predesigned number of (for example 5 or more) quantum dots are used to label, detect, and quantify the molecular targets.

Previous fluorescent in situ hybridizations done with quantum dots labeled probes relied on imaging only one quantum dot molecule per oligonucleotide (Yang, H., Wanner, I. B., Roper, S. D., and Chaudhari, N. (1999). An optimized method for in situ hybridization with signal amplification that allows the detection of rare mRNAs. J. Histochem. Cytochem. 47, 431-446; Chan, P., Yuen, T., Ruf, F., Gonzalez-maeso, J., & Sealfon, S. C. (2005). Method for multiplex cellular detection of mRNAs using quantum dot fluorescent in situ hybridization. Nucleic acids research, 33(18), 1-8. doi:10.1093/nar/gni162; Choi, Y., Kim, H. P., Hong, S. M., Ryu, J. Y., Han, S. J., & Song, R. (2009). In situ Visualization of Gene Expression Using Polymer-Coated Quantum-Dot—DNA Conjugates. Small, 5(18), 2085-2091. doi:10.1002/smll.200900116; Akita, H., Umetsu, Y., Kurihara, D., & Harashima, H. (2011). Dual imaging of mRNA and protein production: An investigation of the mechanism of heterogeneity in cationic lipid-mediated transgene expression. International Journal of Pharmaceutics, 415, 218-220. doi:10.1016/j.ijpharm.2011.05.051; Ioannou, A., Eleftheriou, I., Lubatti, A., Charalambous, A., & Skourides, P. A. (2012). High-resolution whole-mount in situ hybridization using Quantum Dot nanocrystals. Journal of biomedicine & biotechnology, 2012, 627602. doi:10.1155/2012/627602). This strategy is not effective to image targeted single ribonucleic acids because quantum dots have the intrinsic property of emitting fluorescent intermittently, thus showing an on/off emission state upon light excitation (Wu, S., Zhao, X., Zhang, Z., and Xie, H. (2006). Quantum-Dot-Labeled DNA Probes for Fluorescence In Situ Hybridization (FISH) in the Microorganism Escherichia coli. 1062-1067; Smith, A. M., Duan, H., Mohs, A. M., and Nie, S. (2008). Bioconjugated quantum dots for in vivo molecular and cellular imaging. Adv. Drug Deliv. Rev. 60, 1226-1240). This phenomenon, so called blinking, has been observed at different intervals at the order of deciseconds (Wu, S., Zhao, X., Zhang, Z., and Xie, H. (2006). Quantum-Dot-Labeled DNA Probes for Fluorescence In Situ Hybridization (FISH) in the Microorganism Escherichia coli. 1062-1067; Friedrich, M., Nozadze, R., Gan, Q., Zelman-femiak, M., Ermolayev, V., Wagner, T. U., and Harms, G. S. (2009). Biochemical and Biophysical Research Communications Detection of single quantum dots in model organisms with sheet illumination microscopy. Biochem. Biophys. Res. Commun. 390, 722-727), and make the use of one quantum dot impractical for the steady imaging of single molecule ribonucleic acids hybridized with one probe. As a consequence, one molecule may or may not be imaged upon the light excitation, and the accurate counting of ribonucleic molecules is compromised.

The current imaging and detection of single molecules of ribonucleic acid relies on the use of approximately 40 probes (20 nucleotides long) labeled with organic dyes. In some embodiments, a small predesigned number (for example 5 or more) probes labeled with quantum dots are used to achieve single ribonucleic acid molecule imaging. In some embodiments, the probes can be about 30 nucleotides long but any length compatible with the uses described herein may be used. In some embodiments, the probes are between about 10, about 20, about 30, about 40, about 50, about 60, about 70, about 80, about 90 or about 100 nucleotides in length, or any other length between two aforementioned values. In some embodiments, the methods reduce the cost of single molecule detection by several fold, compared to currently available methods (such as organic dye-based methods). The cost estimates of oligonucleotides synthesis obtained from two companies, showed that, in some embodiments, the method can reduce the cost of oligonucleotide synthesis approximately 5 fold per target of ribonucleic acid (See Table 2 below for values).

TABLE 2
Cost of oligonucleotide synthesis estimated from two independent
companies. Note that the difference between the costs is approximately
5 fold between biotin labeling of 5 oligos and amino labeling of 40 oligos.
Company A
scale 100 nmol

Amino labeling of 40 oligos

| N oligos × | length (bases) × | USD/base = | cost of oligos |
|---|---|---|---|
| 40 | $20.00 | $0.55 | $440.30 |
| N oligos × | amino modification × | = | cost of labeling |
| 40 | $28.00 | | $1,120.00 |
| | | total (USD) | $1,560.00 |

Biotin labeling of 5 oligos

| | | | |
|---|---|---|---|
| N oligos × | length (bases) × | USD/base = | cost of oligos |
| 5 | 30 | $0.55 | $82.50 |
| N oligos × | biotin modification × | = | cost of labeling |
| 5 | $45.00 | | $225.00 |
| | | total (USD) | $307.50 |

Company B
scale 50 nmol

Amino labeling of 40 oligos

| | | | |
|---|---|---|---|
| N oligos × | length (bases) × | USD/base = | cost of oligos |
| 40 | 20 | $0.40 | $320.00 |
| N oligos × | amino modification × | = | cost of labeling |
| 40 | $20.00 | | $800.00 |
| | | total (USD) | $1,120.00 |

Biotin labeling of 5 oligos

| | | | |
|---|---|---|---|
| N oligos × | length (bases) × | USD/base = | cost of oligos |
| 5 | 30 | $0.40 | $60.00 |
| N oligos × | biotin modification × | = | cost of labeling |
| 5 | $35.00 | | $175.00 |
| | | total (USD) | $235.00 |

In some embodiments, the method can detect smaller RNAs than the organic dye based approach. This is because the organic dye based approach requires the RNA to be long enough for simultaneous hybridization of 40 oligos, whereas some embodiments described herein, enable detection of shorter RNAs due the need of hybridization of only 2 or more probes. In some embodiments, the method can detect small RNAs, wherein the size of the small RNAs are about 10 nt, about 20 nt, about 30 nt, about 40 nt, about 50 nt, about 60 nt, about 70 nt, about 80 nt, about 90 nt, about 100 nt, about 110 nt, about 120 nt, about 130 nt, about 140 nt, about 150 nt, about 160 nt, about 170 nt, about 180 nt, about 190 nt or about 200 nt in length or any other length between any two aforementioned values.

In some embodiments, the present methods use photo-stable inorganic fluorescent dyes (quantum dots), with fewer probes to achieve imaging and detection of single molecule ribonucleic acids at lower cost compared to other methods.

The current state of the art of the targeted detection of ribonucleic acids by fluorescent in situ hybridization using quantum dots (Yang, H., Wanner, I. B., Roper, S. D., and Chaudhari, N. (1999). An optimized method for in situ hybridization with signal amplification that allows the detection of rare mRNAs. J. Histochem. Cytochem. 47, 431-446; Chan, P., Yuen, T., Ruf, F., Gonzalez-maeso, J., & Sealfon, S. C. (2005). Method for multiplex cellular detection of mRNAs using quantum dot fluorescent in situ hybridization. Nucleic acids research, 33(18), 1-8. doi:10.1093/nar/gni162; Choi, Y., Kim, H. P., Hong, S. M., Ryu, J. Y., Han, S. J., & Song, R. (2009). In situ Visualization of Gene Expression Using Polymer-Coated Quantum-Dot—DNA Conjugates. Small, 5(18), 2085-2091. doi:10.1002/smll.200900116; Akita, H., Umetsu, Y., Kurihara, D., & Harashima, H. (2011). Dual imaging of mRNA and protein production: An investigation of the mechanism of heterogeneity in cationic lipid-mediated transgene expression. International Journal of Pharmaceutics, 415, 218-220. doi:10.1016/j.ijpharm.2011.05.051; Ioannou, A., Eleftheriou, I., Lubatti, A., Charalambous, A., & Skourides, P. A. (2012). High-resolution whole-mount in situ hybridization using Quantum Dot nanocrystals. Journal of biomedicine & biotechnology, 2012, 627602. doi:10.1155/2012/627602) do not achieve or even come close to achieving the detection at a single molecule or single cell resolution. These methods were intended as a coarse quantification of the overall signal of many cells and many RNAs as a whole. These methods suffer from large technical variability and thus are not regarded as promising for precise quantifications.

The current state of the art of single molecule ribonucleotide targeted detection is based on the use of several short oligonucleotides (40 to 48 oligos, 20 nucleotides long) coupled with organic dyes (for example Cy3, Cy5 or other commercial alternatives) (Raj, A., van den Bogaard, P., Rifkin, S. A., van Oudenaarden, A., & Tyagi, S. (2008). Imaging individual mRNA molecules using multiple singly labeled probes. Nature methods, 5(10), 877-9. doi:10.1038/nmeth.1253; Batish, M., Raj, A., & Tyagi, S. (2011). Single molecule imaging of RNA in situ. Methods in molecular biology (Clifton, N. J.), 714, 3-13. doi:10.1007/978-1-61779-005-8_1; Raj, A., & Tyagi, S. (2010). Detection of individual endogenous RNA transcripts in situ using multiple singly labeled probes. Methods in enzymology, 472, 365-86. doi:10.1016/S0076-6879(10)72004-8; Shaffer, S. M., Wu, M.-T., Levesque, M. J., & Raj, A. (2013). Turbo FISH: A Method for Rapid Single Molecule RNA FISH. PloS one, 8(9), e75120. doi:10.1371/journal.pone.0075120).

Some embodiments of the present methods use a pre-designed number of (for example 5 or more) different nucleic acid probes coupled to quantum dots. In some embodiments, each probe is designed and synthesized with a sequence of 30 nucleotides complementary to the target ribonucleic acid. In some embodiments, the custom probes were synthesized with a biotin linked at the 5 prime end of each nucleotide. Nonetheless, it will be appreciated that the biotin linker may be positioned at any location in the probe. For example, in some embodiments, the biotin linker can be attached at the 5′ and/or 3′ end of the probes. In some embodiments, the probe further comprises a nucleic acid linker, wherein the linker is not complementary to the target binding site, wherein the nucleic acid linker comprises about 3, about 4, about 5, about 6, about 7, about 8, about 9 or about 10 nucleic acids or any number of nucleic acids in between any two of the aforementioned values, and wherein the nucleic acid linker is covalently bound to the quantum dot. In some embodiments the quantum dot emits at a high emission wavelength of about 450 nm, about 500 nm, about 550 nm, about 600 nm, about 650 nm, about 700 nm, about 750 nm, about 800 nm or about 850 nm, or any other high emission wavelength between any two aforementioned values. In some embodiments, the quantum dot comprises a size from about 1, about 2, about 3, about 4, about 5, about 6, about 7, about 8, about 9, about 10 or about 11 nanometers or any other size in between any two aforementioned values.

In some of the methods described herein, the quantum dots coated with streptavidin were purchased from a company. The quantum dots were added to a micro tube containing the mixture of oligonucleotides. Experiments were conducted at the ratio of 1 μM of quantum dots per 0.5 μM of oligonucleotides. Nonetheless, it will be appreciated that each composition of probes mixed with quantum dots may be tested for appropriate ratios. Conjugation was conducted at room temperature (24° C.) for 30 minutes. The solution of quantum dots and oligonucleotides were mixed with appropriate buffer for hybridization and the solution was placed in contact with the cover glass containing adhered cells. Two different protocols of fixation and permeabilization strategies for fluorescent in situ hybridization were tested with conjugated probes. The hybridization was successful with either: a) cells fixed with paraformaldehyde and permeabilized with triton 1×; or b) cells fixed and permeabilized with methanol.

As described herein, using some embodiments of the present methods, single molecule ribonucleic acids of the Actin beta gene have been effectively detected in mouse in vitro cultured stem cells by fluorescent in situ hybridizations using five oligonucleotides coupled with quantum dots.

Single-molecule RNA-FISH is an increasingly popular technology that is used in many labs. A major drawback of this technology is its daunting cost, which is approximately $1,500 a gene. It is difficult for a lab to study dozens or hundreds of genes with such cost. In some embodiments, the present methods reduce the cost to less than $500 a gene and may be used in the fast growing market of single-molecule RNA-FISH market.

Single-cell RNA quantification is a fast growing market. First, there is a clear re-direction of government expenditure to single cell analyses. NIH alone plans to initially invest more than $90 million on single cell analyses in 2012-2017 (www.nih.gov/news/health/oct2012/nibib-15.htm). NIH Director's office created a common fund for single cell projects (www.commonfund.nih.gov/singlecell/), which implies larger investments to come. From the industrial sectors, single cell analyses are also on the rise to cancer, stem cell, and neural analyses. After all, cell heterogeneity is a central issue of these analyses. These fast growing interests will created a high demand for probes for single molecule RNA detection.

The majority of the scientific discoveries of a given gene are obtained by studying its expression. The information obtained from gene transcription is used to infer important parameters related to gene functions, from the structure of the coding sequence, all the way to the potential interactions at the protein level. While the majority of the information of gene expression has been collected from cell populations (for example: cell culture, or tissue samples), an expansive area or research has directed the attention for the individual cells. Recent recognition of cell heterogeneity in normal and diseased tissues further highlighted the importance of accurate RNA detection and quantification in single cells.

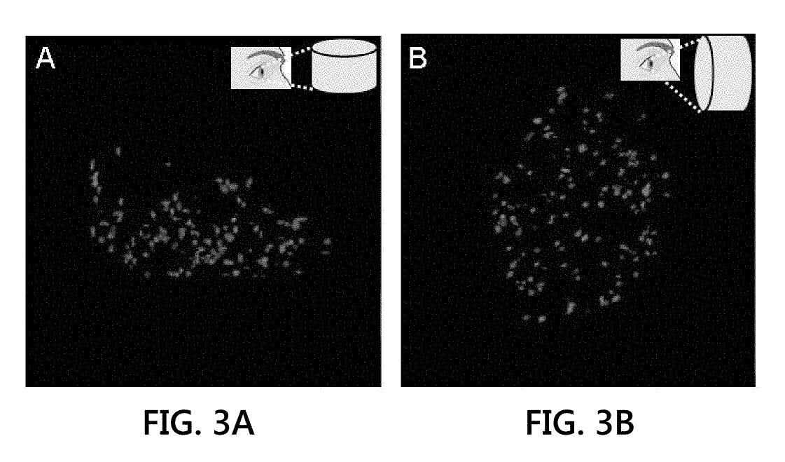

Accurate quantification of gene transcription brings along technical challenges. The majority of available methods are designed to approximate the copy number (counts) of the transcripts of a gene, including real-time qPCR (Valleron W, Laprevotte E, Gautier E F, Quelen C, Demur C, et al. (2012) Specific small nucleolar RNA expression profiles in acute leukemia. Leukemia 26: 2052-2060), gene expression microarrays (Schena M, Shalon D, Davis R W, Brown P O (1995) Quantitative monitoring of gene expression patterns with a complementary DNA microarray. Science 270: 467-470), and RNA sequencing (Chu Y, Corey D R (2012) RNA sequencing: platform selection, experimental design, and data interpretation. Nucleic Acid Ther 22: 271-274). Technical biases were inevitably introduced in during the biochemical or biophysical steps of approximation. Additionally, RNA quantification at single cell level poses further challenges. Most methods for single cell analysis require amplification of RNA before measurement, including Nanostring nCounter (see FIG. 3 in (Nanostring (2014) nCounter Single Cell Gene Expression)), SMART-seq (Ramskold D, Luo S, Wang Y C, Li R, Deng Q, et al. (2012) Full-length mRNA-Seq from single-cell levels of RNA and individual circulating tumor cells. Nat Biotechnol 30: 777-782), and Fluidigm C1-BioMark (Moignard V, Macaulay I C, Swiers G, Buettner F, Schutte J, et al. (2013) Characterization of transcriptional networks in blood stem and progenitor cells using high-throughput single-cell gene expression analysis. Nat Cell Biol 15: 363-372). The RNA amplification step adds to the technical biases in these measurement methods. To our knowledge, there is only one technique that can reliably count RNA copy numbers in single cells (Raj A, Peskin C S, Tranchina D, Vargas D Y, Tyagi S (2006) Stochastic mRNA synthesis in mammalian cells. PLoS biology 4: e309). The single molecule RNA fluorescent in situ hybridization (smRNA-FISH) technology visualizes every RNA molecule of a gene, which enables directly counting RNA molecules (Batish M, Raj A, Tyagi S (2011) Single molecule imaging of RNA in situ. Methods in molecular biology (Clifton, N J) 714: 3-13: Raj A, van den Bogaard P, Rifkin S A, van Oudenaarden A, Tyagi S (2008) Imaging individual mRNA molecules using multiple singly labeled probes. Nature methods 5: 877-879; Shaffer S M, Wu M-T, Levesque M J, Raj A (2013) Turbo FISH: A Method for Rapid Single Molecule RNA FISH. PloS one 8: e75120). This key feature differentiates this digital technology (smRNA-FISH) from other approximation techniques. Moreover, smRNA-FISH does not need amplification to quantify RNA molecules in single cells, eliminating a major source of technical bias in single cell analysis.

A major limitation of standard smRNA-FISH is the requirement of a large number of oligonucleotide probes. The technique requires approximately 40 oligonucleotide probes per gene, so as to attach ˜40 organic fluorescent molecules (dyes) to every RNA molecule, which accumulates a signal that is clearly above background. Under conventional instrumentation (fluorescent microscopes), this technology cannot work with fewer probes, due to the difficulty to separate signals from noises. The probe number can be reduced provided that the instrument for super-resolution imaging is available (Lubeck E, Cai L (2012) Single-cell systems biology by super-resolution imaging and combinatorial labeling. Nat Methods 9: 743-748). However, such instrument costs about US $500,000, and is not available in the majority of research labs. Hereafter, we only discuss in the context of typical fluorescent microscopes as the available instrument. In some embodiments described herein, fluorescent microscopes are used in the detection of target nucleic acid. In some embodiments described herein the method further comprises single molecule analysis of RNA splicing and RNA-RNA interaction in vivo.

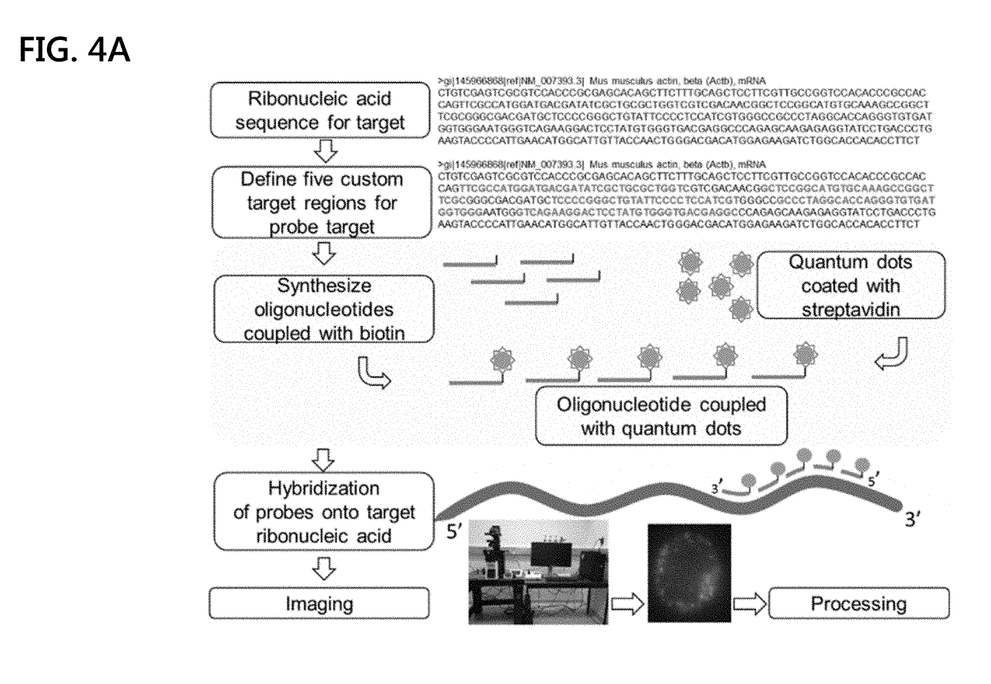

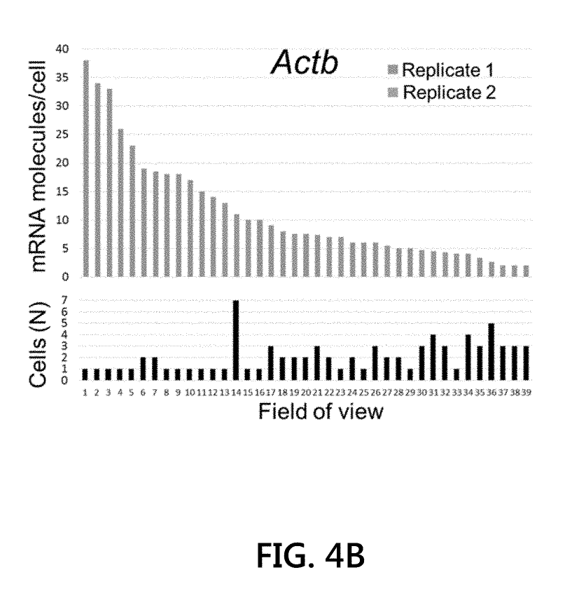

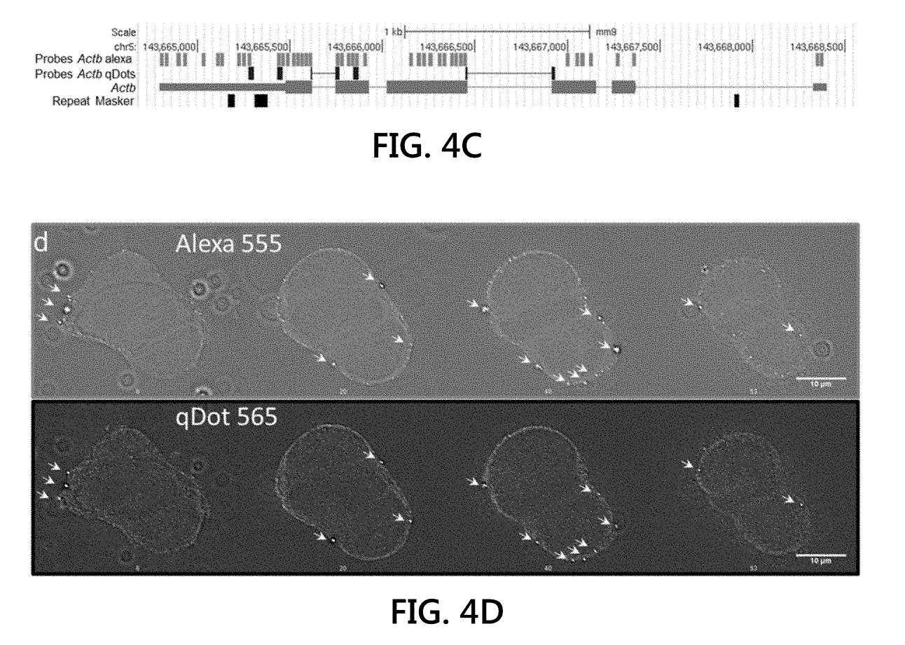

The requirement of large number of probes posed several drawbacks. First, standard smRNA-FISH cannot assay relatively small RNA molecules or specifically target a small region on RNA molecules. The 40 or so probes have to be non-overlapping in order to allow simultaneous attachment to the target, which requires a minimal length of 1000 bases of the target sequence. However, more than 50% human mRNA molecules are shorter than 1000 bases (see FIG. 4 in Takahashi H, Lassmann T, Murata M, Carninci P (2012) 5′ end-centered expression profiling using cap-analysis gene expression and next-generation sequencing. Nat Protoc 7: 542-561)). Moreover, approximately 75% of human lncRNAs are shorter than 1000 base; snoRNAs and tRNAs are typically between 70 to 95 bases, and snRNAs are approximately 150 bases. Taken together, the human transcriptome is primarily composed of RNA molecules with lengths between 70 to 1000 bases, which cannot be assayed by standard smRNA-FISH. Even if a RNA molecule is longer than 1000 bases, it is unlikely that the entire transcript is available for hybridization. Various parts of an RNA molecule could have been bound by proteins (Ray D, Kazan H, Cook K B, Weirauch M T, Najafabadi H S, et al. (2013) A compendium of RNA-binding motifs for decoding gene regulation. Nature 499: 172-177) or tightly bundled (Tripathi V, Ellis J D, Shen Z, Song D Y, Pan Q, et al. (2010) The nuclear-retained noncoding RNA MALAT1 regulates alternative splicing by modulating SR splicing factor phosphorylation. Mol Cell 39: 925-938), and thus greatly reduce the portion available for probe hybridization. Moreover, because the protein-bound portions of an RNA molecule are often unknown, the failure of hybridizing a fraction of the probes could result in false negatives. For the same reason, the technique cannot specifically investigate a specific region (<1000 bases) of an RNA molecule. As a result, standard smRNA-FISH cannot be used to investigate RNA-RNA interactions, or RNA splicing. In some embodiments herein, a method for detecting at least one target nucleic acid is provided, wherein the method comprises contacting said at least one target nucleic acid with a plurality of different nucleic acid probes which are associated with at least one detectable component with a high wavelength emission under conditions in which said plurality of different nucleic acid probes bind to said at least one target nucleic acid. In some embodiments, the one target nucleic acid is an RNA. In some embodiments, the RNA comprises 70 to 1000 bases. In some embodiments, the RNA comprises 70, 80, 90, 100, 200, 300, 400, 500, 600, 700, 800, 900 or 1000 bases or any number of bases between any two aforementioned values. In some embodiments, the method further comprises single molecule analysis of RNA splicing and RNA-RNA interaction in vivo.

Second, the cost for synthesizing 40 oligonucleotides (25 base each) is approximately US $370 (per gene). The cost would ramp up quickly for assaying multiple genes.

Third, it is difficult to assay multiple genes in parallel. This is due to two reasons. A different fluorescent dye is needed for each gene. However, the different organic dyes exhibit vastly different photo stabilities (Lichtman J W, Conchello J A (2005) Fluorescence microscopy. Nat Methods 2: 910-919; Panchuk-Voloshina N, Haugland R P, Bishop-Stewart J, Bhalgat M K, Millard P J, et al. (1999) Alexa dyes, a series of new fluorescent dyes that yield exceptionally bright, photostable conjugates. J Histochem Cytochem 47: 1179-1188), making it difficult to obtain signals at a consistent level. The emission of organic dyes typically follow long tailed distributions, making it difficult to eliminate the contamination (leak) of signals from another dye. It is even more difficult to solve the two problem altogether, which is required to assay multiple genes in parallel. Although two-color sm RNA-FISH have been attempted (ref), we are not aware of any successes in accurately counting the transcript copy numbers of multiple genes in parallel. Additionally, there are concerns of leakage and different degrees of photo stability.

Some embodiments described herein through QD-smRNA-FISH using hybridization of quantum dot-labeled single stranded DNA oligonucleotides to the RNA targets for single molecule detection and counting. In some embodiments herein, methods for QD-smRNA-FISH are provided, wherein the method comprises hybridizing quantum dot-labeled single stranded DNA oligonucleotides to RNA targets for single molecule detection and counting. Quantum dots are inorganic dyes with greater photo-stability and greater fluorescence emission compared to organic dyes (Resch-Genger U, Grabolle M, Cavaliere-Jaricot S, Nitschke R, Nann T (2008) Quantum dots versus organic dyes as fluorescent labels. Nature methods 5: 763-775). To our knowledge, this is the first time that quantum dots have been used in counting single molecules. It is also the first time that quantum dots have been applied to visualize individual RNA molecules. Although quantum dots were used to image protein molecules (Zrazhevskiy P, Gao X (2013) Quantum dot imaging platform for single-cell molecular profiling. Nat Commun 4: 1619; Smith A M, Nie S (2012) Compact quantum dots for single-molecule imaging. J Vis Exp.), it is commonly accepted that quantum dots are not applicable to count single molecules. This is due to the well-known ‘blinking’ problem, that the signals are intermittent (Medintz I L, Uyeda H T, Goldman E R, Mattoussi H (2005) Quantum dot bioconjugates for imaging, labelling and sensing. Nat Mater 4: 435-446; Roch N, Florens S, Bouchiat V, Wernsdorfer W, Balestro F (2008) Quantum phase transition in a single-molecule quantum dot. Nature 453: 633-637). If quantum dots were used to label single molecules, at any time of imaging, a random subset of quantum dots would be invisible, making a random subset of single molecules undetectable.

In some embodiments described herein, methods for QD-smRNA-FISH are provided, wherein the method comprises hybridizing quantum dot-labeled single stranded DNA oligonucleotides to RNA targets for single molecule detection and counting. In some embodiments, the quantum dots are used for counting single molecules. In some embodiments, quantum dots are applied to visualize individual RNA molecules. In some embodiments, the at least one quantum dot or comparable particle characterized by a high wavelength emission emits at a high emission wavelength of about 450 nm, about 500 nm, about 550 nm, about 600 nm, about 650 nm, about 700 nm, about 750 nm, about 800 nm or about 850 nm, or any other high emission wavelength between any two aforementioned values. In some embodiments, the quantum dot comprises a size from about 1, about 2, about 3, about 4, about 5, about 6, about 7, about 8, about 9, about 10 or about 11 nanometers or any other size in between any two aforementioned values. In some embodiments, the method further comprises single molecule analysis of RNA splicing and RNA-RNA interaction in vivo.

An idea was developed to solve this problem. The idea is that by attaching several quantum dots to each target molecule, the complementation of their signals should compensate the interrupted signals of any single quantum dots. Thus, the accumulative signal for the target molecule would be continuous. An image acquisition at any time would not lose any molecules. In a validation experiment described in an embodiment herein, QD-smRNA-FISH with 5 probes identified the same RNA molecules as a standard smRNA-FISH with 43 probes. The reduction of the number of required probes enables assaying the RNAs between 80-1000 bases, which cannot be assayed by standard RNA-FISH. Thus unexpected advantages were obtained with the forgoing QD-smRNA FISH methodology. It also enables single molecule analysis of RNA splicing and RNA-RNA interaction in vivo. Furthermore, QD-smRNA-FISH offers a simple method to analyze multiple genes in parallel. The last but not the least, it reduces the reagent cost very several fold, because oligo synthesis is the cost.

In some embodiments, a method for detecting at least one target nucleic acid is provided, wherein the method comprises contacting said at least one target nucleic acid with a plurality of different nucleic acid probes which are associated with at least one detectable component with a high wavelength emission under conditions in which said plurality of different nucleic acid probes bind to said at least one target nucleic acid. In some embodiments, the method further comprises single molecule analysis of RNA splicing and RNA-RNA interaction in vivo.

Interactions among multiple levels of molecular properties has brought great interest onto the existence, albeit poorly understood, functions of RNA-RNA interactions. This increasing pursuit of RNA-RNA interactions has led researchers to develop important tools to characterize miRNA-RNA interactions (Chi S W, Zang J B, Mele A, Darnell R B (2009) Argonaute HITS-CLIP decodes microRNA-mRNA interaction maps. Nature 460: 479-486; Helwak A, Kudla G, Dudnakova T, Tollervey D (2013) Mapping the human miRNA interactome by CLASH reveals frequent noncanonical binding. Cell 153: 654-665). Despite limited evidence (Batista P J, Chang H Y (2013) Long noncoding RNAs: cellular address codes in development and disease. Cell 152: 1298-1307), it has been hypothesized that long non coding RNAs (lncRNA) interact with protein coding RNAs (mRNA) for post transcription regulation of gene expression (Amaral P P, Dinger M E, Mercer T R, Mattick J S (2008) The eukaryotic genome as an RNA machine. Science 319: 1787-1789). To that end, computational models have provided tools for hypothesis driven prediction of lncRNA-mRNA pairs (Kato Y, Sato K, Hamada M, Watanabe Y, Asai K, et al. (2010) RactIP: fast and accurate prediction of RNA-RNA interaction using integer programming. Bioinformatics 26: i460-466; Wenzel A, Akbasli E, Gorodkin J (2012) RIsearch: fast RNA-RNA interaction search using a simplified nearest-neighbor energy model. Bioinformatics 28: 2738-2746), yet no experimental approach has been devised to validate RNA-RNA interactions. Using the principle that interacting RNAs are physically proximal of each other in the cell, it is proposed that the embodiments described herein can be used to bridge the gap between computational predictions and experimental evidence of RNA-RNA interactions by applying QD-smRNA-FISH to image and visualize RNA-RNA interactions.

### Results

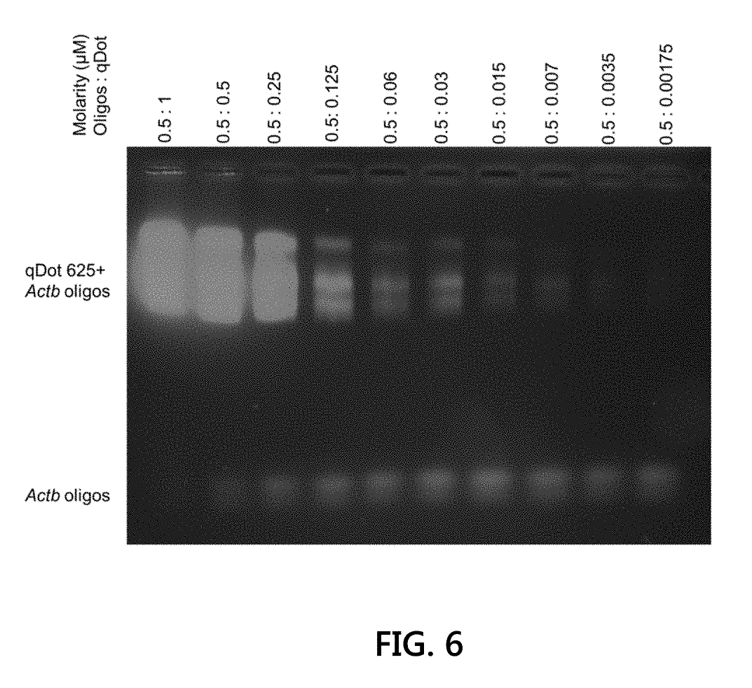

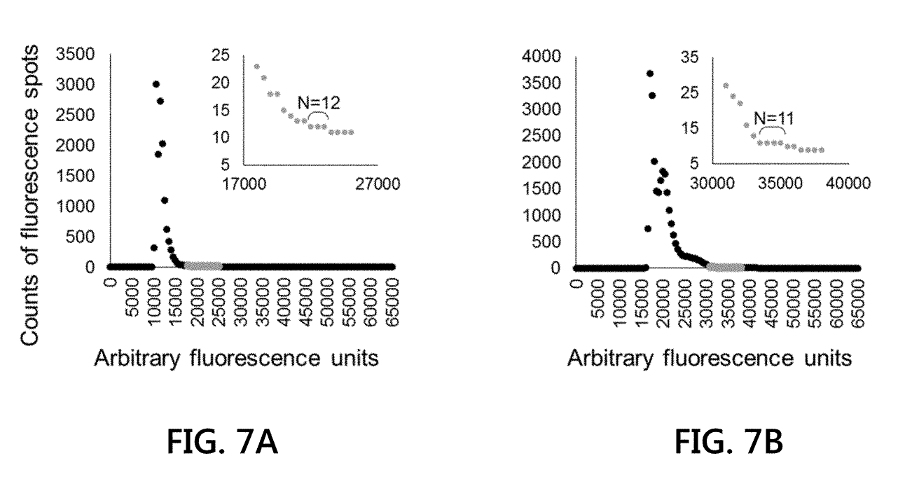

Development of the QD-smRNA-FISH technique. Multiple QD-labeled probes targeting a particular RNA of interest were designed (FIG. 4A). The sequence of the target mRNA is provided in SEQ ID NO: 59 (See also Genbank Accession No. gi 145966868, the disclosure of which is incorporated herein by reference in its entirety). For all QD-smRNA-FISH experiments in this paper, five oligonucleotides per gene were used. Each oligonucleotide was designed to be 25-30 bases and synthesized to contain one biotin attached to the 5′ end (IDT, Table 2). As QDs were coated with streptavidin (Invitrogen), labeling was achieved at room temperature for 30 minutes at ratio of 0.5 μM of oligonucleotides per 1 μM of QDs (FIG. 6). The hybridization and imaging protocol for QD-smRNA-FISH was optimized by testing a number of variations of reagents and parameters from the hybridization protocol of standard smRNA-FISH (Shaffer S M, Wu M-T, Levesque M J, Raj A (2013) Turbo FISH: A Method for Rapid Single Molecule RNA FISH. PloS one 8: e75120) (Methods, Supplementary Text). After probe hybridization and imaging, raw images with were processed ImageJ (Schneider C A, Rasband W S, Eliceiri K W (2012) NIH Image to ImageJ: 25 years of image analysis. Nature methods 9: 671-675) by first applying the Laplacian of Gaussian filter (Sage D, Neumann F R, Hediger F, Gasser S M, Unser M (2005) Automatic tracking of individual fluorescence particles: application to the study of chromosome dynamics. IEEE transactions on image processing: a publication of the IEEE Signal Processing Society 14: 1372-1383), and then counting the 3D rendered signal spots (Bolte S, Cordelières F P (2006) A guided tour into subcellular colocalization analysis in light microscopy. Journal of microscopy 224: 213-232) (Methods). It is observed that the final counts were insensitive to the threshold used (FIG. 7A and FIG. 7B).

TABLE 3
Oligonucleotides used in QD-smRNA-FISH experiments.

| Gene | | | | |
|---|---|---|---|---|
| symbol | Accession | Attachment | Oligonucleotide 5′- 3′ | CG % |
| Actb | NM_007393.3 | 5′ Biotin | TCTCAAACATGATCTGGGTCATCTTTTCAC | 40.0 |
| | | | (SEQ ID NO: 1) | |
| Actb | NM_007393.3 | 5′ Biotin | GTTGGCATAGAGGTCTTTACGGATGTCAAC | 46.7 |
| | | | (SEQ ID NO: 2) | |
| Actb | NM_007393.3 | 5′ Biotin | CAATGATCTTGATCTTCATGGTGCTAGGAG | 43.3 |
| | | | (SEQ ID NO: 3) | |
| Actb | NM_007393.3 | 5′ Biotin | TTTTGTCAAAGAAAGGGTGTAAAACGCAGC | 40.0 |
| | | | (SEQ ID NO: 4) | |
| Actb | NM_007393.3 | 5′ Biotin | CGTTCCAGTTTTTAAATCCTGAGTCAAAAG | 36.7 |
| | | | (SEQ ID NO: 5) | |
| Malat1 | NR_002847.2 | 5′ Biotin | AAGGTCTCACATCACACACTCAT | 45.8 |
| | | | (SEQ ID NO: 6) | |
| Malat1 | NR_002847.2 | 5′ Biotin | CAAAGAACAGACATGACCTGAAGT | 41.6 |
| | | | (SEQ ID NO: 7) | |
| Malat1 | NR_002847.2 | 5′ Biotin | TTCTAATAGCAGCAGATTGGAACAG | 40.0 |
| | | | (SEQ ID NO: 8) | |
| Malat1 | NR_002847.2 | 5′ Biotin | AACAACCACTACTCCAAACACTTG | 41.6 |
| | | | (SEQ ID NO: 9) | |
| Malat1 | NR_002847.2 | 5′ Biotin | CTCAACACTCAGCCTGTTACTCAT | 45.8 |
| | | | (SEQ ID NO: 10) | |
| Scl2a3 | NM_011401.4 | 5′ Biotin | TAGCCACAATACAGACAAAGCTCAT | 44 |
| | | | (SEQ ID NO: 11) | |
| Scl2a3 | NM_011401.4 | 5′ Biotin | CTCAAAGAAGGCTACGTAGATCAAG | 47.8 |
| | | | (SEQ ID NO: 12) | |
| Scl2a3 | NM_011401.4 | 5′ Biotin | AGAAAGTTGGAGGTCCAGTTACAA | 40.0 |
| | | | (SEQ ID NO: 13) | |
| Scl2a3 | NM_011401.4 | 5′ Biotin | CGTCCTTGAAGATTCCTGTTGAGTA | 44.0 |
| | | | (SEQ ID NO: 14) | |
| Scl2a3 | NM_011401.4 | 5′ Biotin | CCTATCATATGCAGGGTTCTCCT | 41.6 |
| | | | (SEQ ID NO: 15) | |

QD-smRNA-FISH signals are continuous. The emission intermittency of QD-smRNA-FISH were tested. Five hybridization probes for Actb mRNA were designed (Table 3) and were coupled with QDs. After probe hybridization, 50 images of the same experimental sample were acquired with 1 second interval between any two image acquisitions. To simplify the analysis, each image was acquired only on one focal plane (stack). This makes it a more rigorous test because acquiring multi-stack images would offer greater chances of an RNA molecule to emit signal during the acquisition of any of the stacks. None of the spots was lost in any of the 50 images (systemsbio.ucsd.edu/qdsmrnafish/, the disclosure of which is incorporated herein by reference in its entirety), confirming our theory that the cumulative signal is non-intermittent.

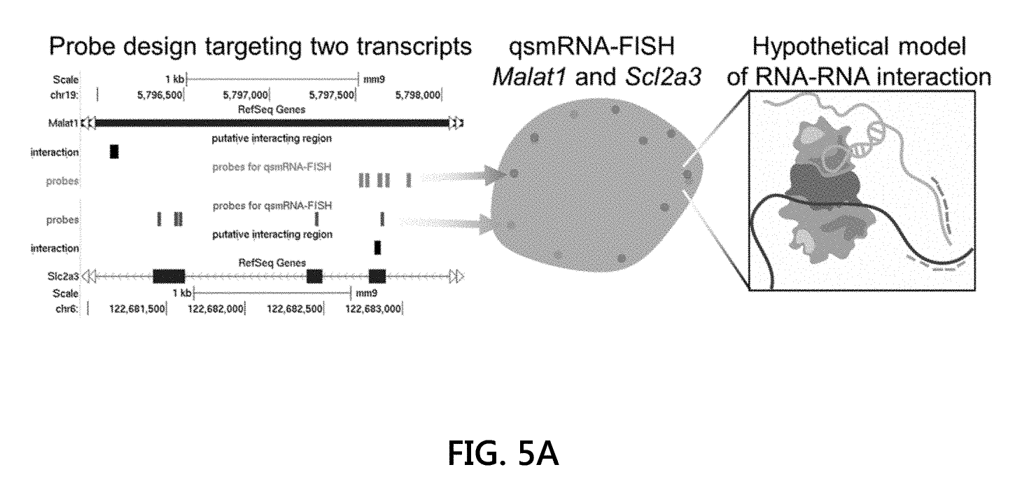

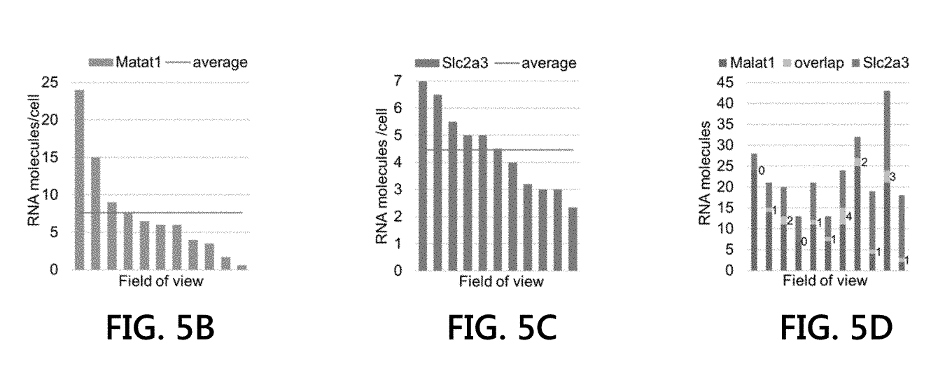

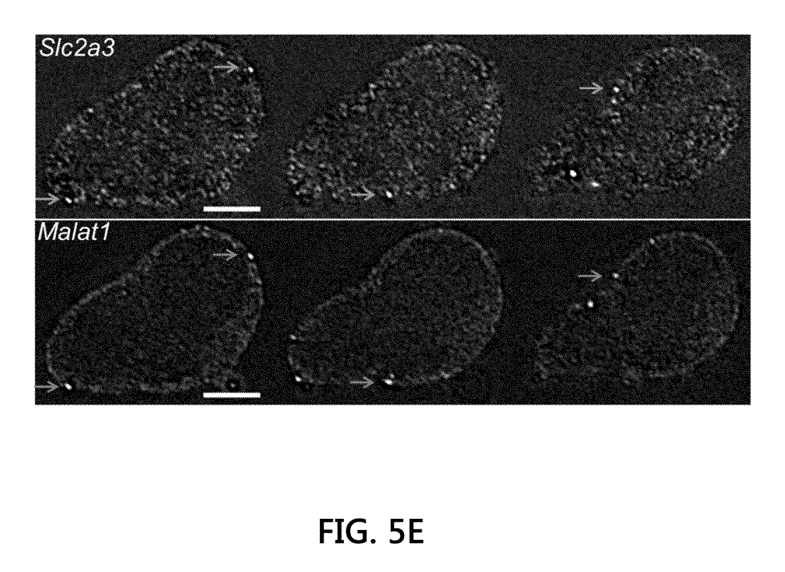

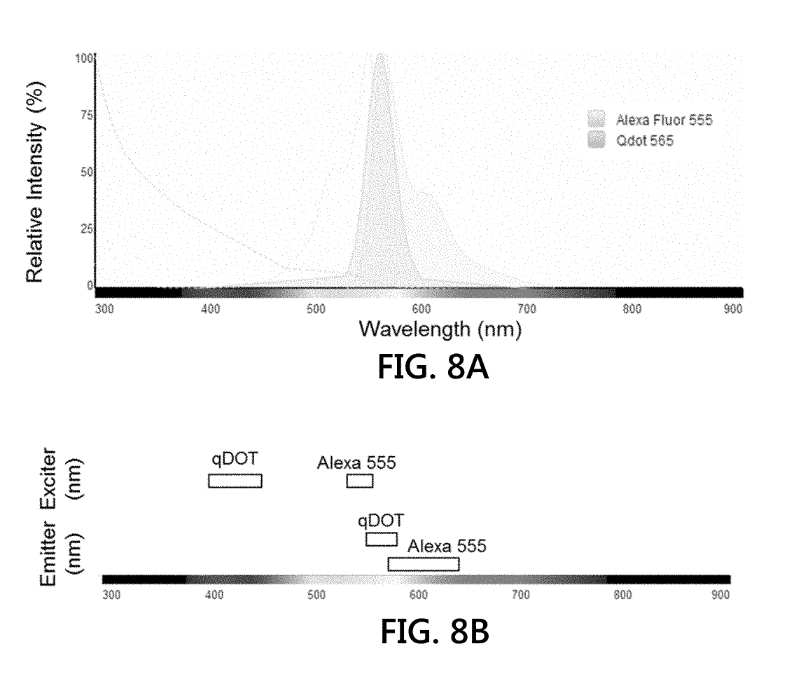

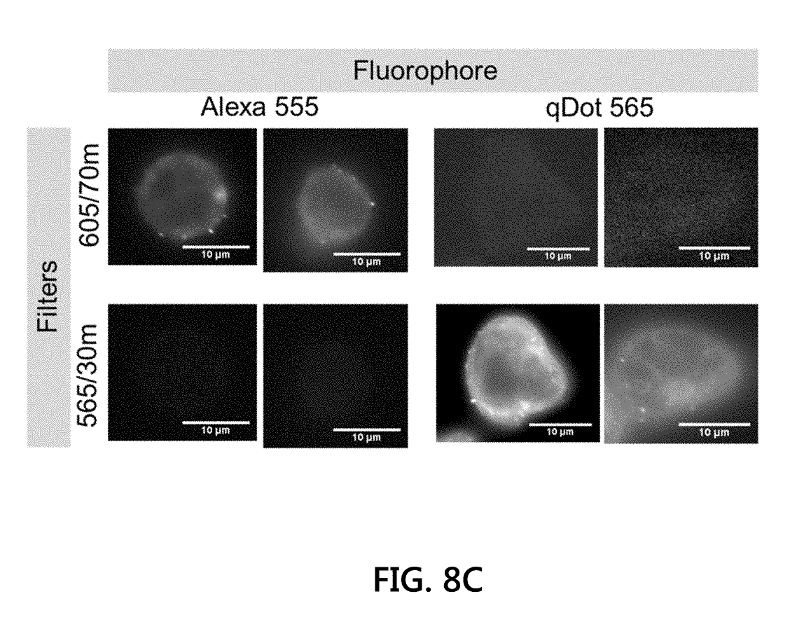

Comparison of QD-smRNA-FISH with standard smRNA-FISH. The ability of QD-smRNA-FISH to quantify RNA molecules was tested. Multi-stack images were acquired on the same hybridized sample as above. Following image processing, 11±9 (mean±standard deviation) Actb mRNA molecules per cell imaged (N=82) (FIG. 4B) were identified. To access the accuracy of probing an RNA molecule with this embodiment, QD-smRNA-FISH, 43 probes were designed and labeled with Alexa555 to compare the co-localization of spots with different set of dyes and probes targeting the same RNA (Table 4, FIG. 4C). QD-smRNA-FISH with five probes, allowed identification of almost all the same spots that 43 probes labeled with Alexa555 identified (FIG. 5D). The observed co-localization could not be resulted from cross imaging of dyes between filters as the dyes were excited at different wavelengths (Methods, Table 5, FIG. 8A to 8C). Thus, QD-smRNA-FISH performed with five probes achieved accuracy equivalent to smRNA-FISH with 43 probes labeled with organic dyes. As such, it can be concluded that the QD-smRNA-FISH leads to a surprising effect of being more efficient when compared to using smRNA-FISH with 43 probes labeled with organic dyes.

TABLE 4
Oligonucleotides used in smRNA-FISH experiments.

| Gene symbol | Accession | Attachment | Oligonucleotide 5′- 3′ | CG % |
|---|---|---|---|---|
| Actb | NM_007393.3 | 5′ Biotin | TGCAAAGAAGCTGTGCTCGC | 55 |
| | | | (SEQ ID NO: 16) | |
| Actb | NM_007393.3 | 5′ Biotin | TGTGGACCGGCAACGAAGGA | 60 |
| | | | (SEQ ID NO: 17) | |
| Actb | NM_007393.3 | 5′ Biotin | ATATCGTCATCCATGGCGAA | 45 |
| | | | (SEQ ID NO: 18) | |
| Actb | NM_007393.3 | 5′ Biotin | ACGATGGAGGGGAATACAGC | 55 |
| | | | (SEQ ID NO: 19) | |
| Actb | NM_007393.3 | 5′ Biotin | CACATAGGAGTCCTTCTGAC | 50 |
| | | | (SEQ ID NO: 20) | |
| Actb | NM_007393.3 | 5′ Biotin | GTACTTCAGGGTCAGGATAC | 50 |
| | | | (SEQ ID NO: 21) | |
| Actb | NM_007393.3 | 5′ Biotin | GTTGGTAACAATGCCATGTT | 40 |
| | | | (SEQ ID NO: 22) | |
| Actb | NM_007393.3 | 5′ Biotin | ACACGCAGCTCATTGTAGAA | 45 |
| | | | (SEQ ID NO: 23) | |
| Actb | NM_007393.3 | 5′ Biotin | TGATCTGGGTCATCTTTTCA | 40 |
| | | | (SEQ ID NO: 24) | |
| Actb | NM_007393.3 | 5′ Biotin | GGGGTGTTGAAGGTCTCAAA | 50 |
| | | | (SEQ ID NO: 25) | |
| Actb | NM_007393.3 | 5′ Biotin | CTGGATGGCTACGTACATGG | 55 |
| | | | (SEQ ID NO: 26) | |
| Actb | NM_007393.3 | 5′ Biotin | AGAGGCATACAGGGACAGCA | 55 |
| | | | (SEQ ID NO: 27) | |
| Actb | NM_007393.3 | 5′ Biotin | CATCACAATGCCTGTGGTAC | 50 |
| | | | (SEQ ID NO: 28) | |
| Actb | NM_007393.3 | 5′ Biotin | TCGTAGATGGGCACAGTGTG | 55 |
| | | | (SEQ ID NO: 29) | |
| Actb | NM_007393.3 | 5′ Biotin | ATGGCGTGAGGGAGAGCATA | 55 |
| | | | (SEQ ID NO: 30) | |
| Actb | NM_007393.3 | 5′ Biotin | ATCTTCATGAGGTAGTCTGT | 40 |
| | | | (SEQ ID NO: 31) | |
| Actb | NM_007393.3 | 5′ Biotin | CTGTGGTGGTGAAGCTGTAG | 55 |
| | | | (SEQ ID NO: 32) | |
| Actb | NM_007393.3 | 5′ Biotin | TTGATGTCACGCACGATTTC | 45 |
| | | | (SEQ ID NO: 33) | |
| Actb | NM_007393.3 | 5′ Biotin | ATCTCCTGCTCGAAGTCTAG | 50 |
| | | | (SEQ ID NO: 34) | |
| Actb | NM_007393.3 | 5′ Biotin | TAGTTTCATGGATGCCACAG | 45 |
| | | | (SEQ ID NO: 35) | |
| Actb | NM_007393.3 | 5′ Biotin | GGTCTTTACGGATGTCAACG | 50 |
| | | | (SEQ ID NO: 36) | |
| Actb | NM_007393.3 | 5′ Biotin | GACAGCACTGTGTTGGCATA | 50 |
| | | | (SEQ ID NO: 37) | |
| Actb | NM_007393.3 | 5′ Biotin | TGGGTACATGGTGGTACCAC | 55 |
| | | | (SEQ ID NO: 38) | |
| Actb | NM_007393.3 | 5′ Biotin | AGAGCAGTAATCTCCTTCTG | 45 |
| | | | (SEQ ID NO: 39) | |
| Actb | NM_007393.3 | 5′ Biotin | GATCTTCATGGTGCTAGGAG | 50 |
| | | | (SEQ ID NO: 40) | |
| Actb | NM_007393.3 | 5′ Biotin | GCTCAGGAGGAGCAATGATC | 55 |
| | | | (SEQ ID NO: 41) | |
| Actb | NM_007393.3 | 5′ Biotin | CCGATCCACACAGAGTACTT | 50 |
| | | | (SEQ ID NO: 42) | |
| Actb | NM_007393.3 | 5′ Biotin | ACAGTGAGGCCAGGATGGAG | 60 |
| | | | (SEQ ID NO: 43) | |
| Actb | NM_007393.3 | 5′ Biotin | GATCCACATCTGCTGGAAGG | 55 |
| | | | (SEQ ID NO: 44) | |
| Actb | NM_007393.3 | 5′ Biotin | ACTCATCGTACTCCTGCTTG | 50 |
| | | | (SEQ ID NO: 45) | |
| Actb | NM_007393.3 | 5′ Biotin | TAGAAGCACTTGCGGTGCAC | 55 |
| | | | (SEQ ID NO: 46) | |
| Actb | NM_007393.3 | 5′ Biotin | AACGCAGCTCAGTAACAGTC | 50 |
| | | | (SEQ ID NO: 47) | |
| Actb | NM_007393.3 | 5′ Biotin | GGTTTTGTCAAAGAAAGGGT | 40 |
| | | | (SEQ ID NO: 48) | |
| Actb | NM_007393.3 | 5′ Biotin | TTCACCGTTCCAGTTTTTAA | 35 |
| | | | (SEQ ID NO: 49) | |
| Actb | NM_007393.3 | 5′ Biotin | ATGTTTGCTCCAACCAACTG | 45 |
| | | | (SEQ ID NO: 50) | |
| Actb | NM_007393.3 | 5′ Biotin | TCAGCCACATTTGTAGAACT | 40 |
| | | | (SEQ ID NO: 51) | |
| Actb | NM_007393.3 | 5′ Biotin | CCTGTAACCACTTATTTCAT | 35 |
| | | | (SEQ ID NO: 52) | |
| Actb | NM_007393.3 | 5′ Biotin | CTTTTGGGAGGGTGAGGGAC | 60 |
| | | | (SEQ ID NO: 53) | |
| Actb | NM_007393.3 | 5′ Biotin | CACAGAAGCAATGCTGTCAC | 50 |
| | | | (SEQ ID NO: 54) | |
| Actb | NM_007393.3 | 5′ Biotin | AAAAAGGGAGGCCTCAGACC | 55 |
| | | | (SEQ ID NO: 55) | |
| Actb | NM_007393.3 | 5′ Biotin | GACCAAAGCCTTCATACATC | 45 |
| | | | (SEQ ID NO: 56) | |
| Actb | NM_007393.3 | 5′ Biotin | TTGGTCTCAAGTCAGTGTAC | 45 |
| | | | (SEQ ID NO: 57) | |
| Actb | NM_007393.3 | 5′ Biotin | GTGTAAGGTAAGGTGTGCA | 50 |
| | | | (SEQ ID NO: 58) | |

TABLE 5
Specification of cubes used for imaging.

| Exciter | | Emitter | | |
|---|---|---|---|---|
| Wavelength | Bandwidth | Wavelength | Bandwidth | Dye |
| 545 | 25 | 605 | 70 | Alexa 555 |
| 425 | 50 | 525 | 30 | qDot 525 |
| 425 | 50 | 565 | 30 | qDot 565 |
| 425 | 50 | 605 | 30 | qDot 605 |

Testing RNA-RNA interactions with QD-smRNA-FISH. It was hypothesized that the Malat1 lncRNA and Slc2a3 mRNA interact with each other in mouse embryonic stem (mES) cells. Therefore a dual-probing QD-smRNA-FISH experiment targeting both RNAs to test for the co-localization of transcripts of two genes in the cytoplasm was performed (FIG. 5A). 27 E14 mES cells were analyzed and 7.6 and 4.5 molecules of Malat1 and Slc2a3 RNAs (FIG. 5B, FIG. 5C), respectively, were quantified. The ratio of molecules between the transcripts of Malat1/S1c2a3˜1.7 was slightly lower than the observed for qPCR measurements (Malat1/S1c2a3˜5.1). This minor discrepancy was possibly due to qPCR quantification of Malat1 transcripts in the nucleus (Hutchinson J N, Ensminger A W, Clemson C M, Lynch C R, Lawrence J B, et al. (2007) A screen for nuclear transcripts identifies two linked noncoding RNAs associated with SC35 splicing domains. BMC genomics 8: 39), whereas our QD-smRNA-FISH targets transcripts in the cytoplasm. Interestingly, the QD-smRNA-FISH revealed that the abundance of Malat1 in mouse ES cells cytoplasm is highly variable. While the transcript count for Malat1 ranged from 1 to 24, the range for Slc2a3 was 2 to 7 (FIGS. 5B and 5E).

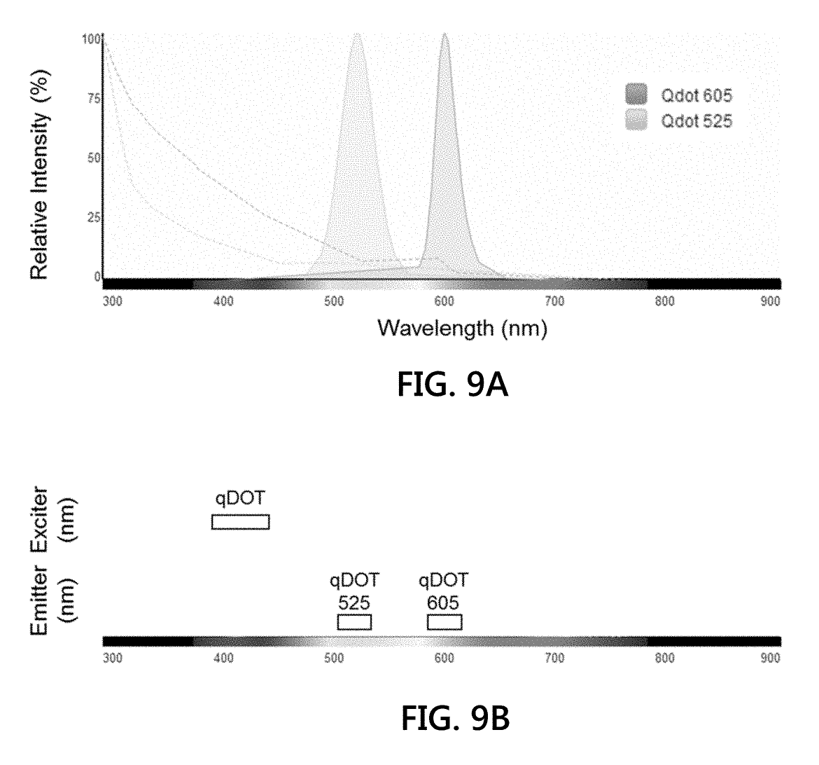

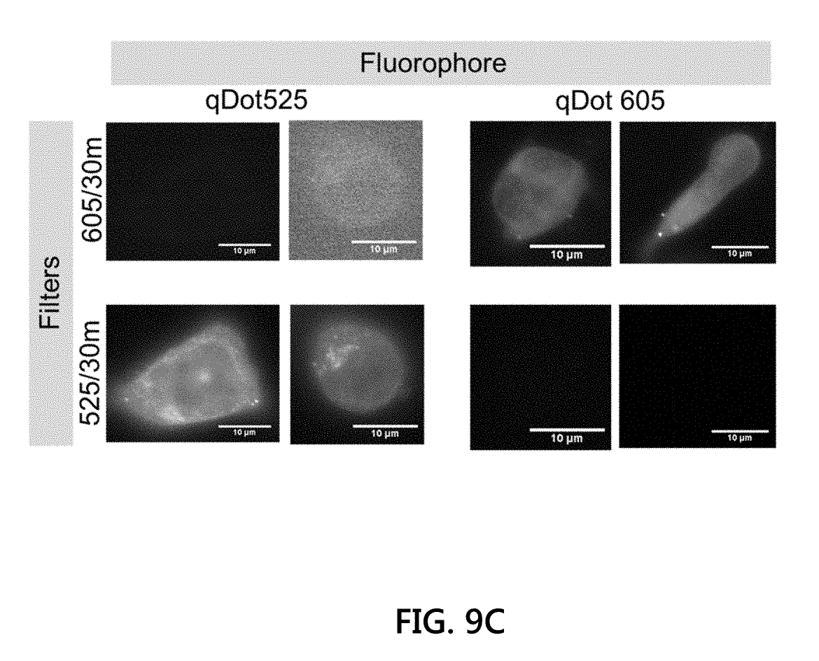

Next, it was questioned whether there were signals showing Malat1 and Slc2a3 hybridizations co-localizing in the same cell. Co-localization of two RNA molecules was determined if the center of mass for two distinct spots, labeled with different dyes, were closer than 2 pixels (214 nm) in each of x, y, z coordinates (FIG. 9A to 9C) (Ghavi-Helm Y, Klein F A, Pakozdi T, Ciglar L, Noordermeer D, et al. (2014) Enhancer loops appear stable during development and are associated with paused polymerase. Nature 512: 96-100.). Ten out of 27 cells expressing Malat1 or Slc2a3 contained overlapping RNA molecules for both genes. Overall, 16 pairs of co-localized RNA molecules were detected (FIG. 5D), ranging from 1 to 3 occurrences in a cell (FIG. 5E). The RNA molecules for both genes showed a random distribution in the cytoplasm, thus it was inferred that the occurrences of two spots labeled with different dyes were not random events (p-value=4×10⁻⁴⁰, hypergeometric test). Therefore, experimental evidence for the interaction of RNA molecules from different genes in a quaternary structure was provided.

### Discussion

The essential idea of this new technique is to exploit the complementation of the random signal intermittencies of a set of quantum dots. The time interval for a quantum dot to stay at the on or the off state can range from milliseconds to seconds (Durisic N, Wiseman P W, Grutter P, Heyes C D (2009) A common mechanism underlies the dark fraction formation and fluorescence blinking of quantum dots. ACS Nano 3: 1167-1175; Yao J, Larson D R, Vishwasrao H D, Zipfel W R, Webb W W (2005) Blinking and nonradiant dark fraction of water-soluble quantum dots in aqueous solution. Proc Natl Acad Sci USA 102: 14284-14289). A quantum dot typically spends more than half the time at the on state (33. Durisic N, Wiseman P W, Grutter P, Heyes C D (2009) A common mechanism underlies the dark fraction formation and fluorescence blinking of quantum dots. ACS Nano 3: 1167-1175; Yao J, Larson D R, Vishwasrao H D, Zipfel W R, Webb W W (2005) Blinking and nonradiant dark fraction of water-soluble quantum dots in aqueous solution. Proc Natl Acad Sci USA 102: 14284-14289; Frantsuzov P A, Volkan-Kacso S, Janko B (2013) Universality of the fluorescence intermittency in nanoscale systems: experiment and theory. Nano Lett 13: 402-408). If it is assumed that the emission intermittency of different quantum dots are independent, at any given time point, the probability of a RNA molecule attached with five quantum dots does not emit fluorescent signal is approximately 0.03 (½⁵). Since a typical image acquisition scans more than 15 focal planes (stacks) at different time points, the probability for an RNA molecule to not be detected in 3 consecutive stacks is in the order of 10⁻⁴-10⁻⁶, and is very small for counting RNA molecules in single cells. If necessary, QD-smRNA-FISH samples can be imaged multiple times, which completely eliminates the probability of a conglomerate of quantum dots to be at the off state. Empirically, any conglomerate to be at the off state even in a single stack in any of the 50 consecutively acquired images was not found (systemsbio.ucsd.edu/qdsmrnafish/, the disclosure of which is incorporated herein by reference in its entirety). Furthermore, QD-smRNA-FISH identified the same targets as smRNA-FISH with continuous signals (FIG. 4C). Taken together, contrary to a common thought, it was theoretically derived and experimentally verified that quantum dots can be applied to counting single molecules.

The major advantage of QD-smRNA-FISH lies in its dependence of less oligonucleotide probes. There is a financial intensive of cost reduction from approximately $370 to $46 per gene. More importantly, it enables targeting the RNA molecules with 1000 or less bases. Such RNA molecules comprise the majority of the RNA, which are too short to be assayed by traditional smRNA-FISH. For the same reason, QD-smRNA-FISH has the unique advantage of targeting specific regions of an RNA molecule. This enables single molecule analysis of RNA splicing and RNA-RNA interactions.

Another advantage of QD-smRNA-FISH is the applicability to assaying the RNA products of multiple genes in parallel. This has been a difficult task for two reasons. First, “while dyes and fluorescent proteins have been the mainstay of fluorescence imaging for decades, their fluorescence is unstable under high photon fluxes necessary to observe individual molecules, yielding only a few seconds of observation before complete loss of signal” (Smith A M, Nie S (2012) Compact quantum dots for single-molecule imaging. J Vis Exp.). The other dyes necessary for targeting different genes could have photo bleached during scanning the first dye (gene). Nanocrystals are more photo stable than organic dyes (Resch-Genger U, Grabolle M, Cavaliere-Jaricot S, Nitschke R, Nann T (2008) Quantum dots versus organic dyes as fluorescent labels. Nature methods 5: 763-775; Lee L Y, Ong S L, Hu J Y, Ng W J, Feng Y, et al. (2004) Use of semiconductor quantum dots for photostable immunofluorescence labeling of Cryptosporidium parvum. Appl Environ Microbiol 70: 5732-5736), allowing for repeated scans of the same experimental sample. Second, organic dyes can contaminate each other, especially when 3 or more different dyes are used together. In contrast, quantum dots have a particular profile of fluorescence spectra, all of them have a narrow Gaussian distribution of the fluorescence intensity (Resch-Genger U, Grabolle M, Cavaliere-Jaricot S, Nitschke R, Nann T (2008) Quantum dots versus organic dyes as fluorescent labels. Nature methods 5: 763-775). This feature forms the basis of multiplexing several quantum dots in one assay. Moreover, the spectral position of emission is tunable according to the particle size (Resch-Genger U, Grabolle M, Cavaliere-Jaricot S, Nitschke R, Nann T (2008) Quantum dots versus organic dyes as fluorescent labels. Nature methods 5: 763-775). As the sizes of the particles used for one specific wave length are very similar, the width of the emission curve is also narrow. For commercial QDs, the majority of the intensity is emitted within a window of 100 nm around the target wave length. This narrow emission of fluorescence of specific QDs allied to the appropriate choice of filters favors the use of QDs in experiments that probe multiple targets with different dyes in the same cell. Experiments have shown dual probing of cells with unambiguous distinction of signal between QDs targeting two different wavelengths (Jaiswal J K, Mattoussi H, Mauro J M, Simon S M (2003) Long-term multiple color imaging of live cells using quantum dot bioconjugates. Nature biotechnology 21: 47-51; Wu X, Liu H, Liu J, Haley K N, Treadway J A, et al. (2003) Immunofluorescent labeling of cancer marker Her2 and other cellular targets with semiconductor quantum dots. Nature biotechnology 21: 41-46). Further multiplexing is possible with QDs targeting three (Han M, Gao X, Su J Z, Nie S (2001) Quantum-dot-tagged microbeads for multiplexed optical coding of biomolecules. Nature biotechnology 19: 631-635) or four (Goldman E R, Clapp A R, Anderson G P, Uyeda H T, Mauro J M, et al. (2004) Multiplexed toxin analysis using four colors of quantum dot fluororeagents. Anal Chem 76: 684-688) wavelengths.

### Methods

Oligonucleotides were synthesized with a biotin attached to their 5′ end (Table 2, Table 3, IDT). Labeling was achieved by incubation of oligonucleotides and dyes coupled with streptavidin at room temperature for 30 minutes at a ratio of 0.5 μM of oligonucleotides per 1 μM of dye (FIG. 6). ES cells were seeded on glass bottom micro-chamber (1.5, Lab Teck) previously coated with poly-d-lysine (5 Sigma) and laminin (0.01 mg/μl, Sigma). Following incubation for 2 hours, cells were washed in nuclease free PBS and permeabilized with methanol at −20° C. FISH experiments were conducted using a modified version of an established smRNA-FISH protocol (Shaffer S M, Wu M-T, Levesque M J, Raj A (2013) Turbo FISH: A Method for Rapid Single Molecule RNA FISH. PloS one 8: e75120). Hybridizations were carried with approximately 15 μM of oligonucleotides in hybridization buffer for 30 minutes at 40° C. Excess of probes and dyes was removed by two washes (SSC 2×, formamide 10%) at 37° C. for 30 minutes. The cells were then imaged in SSC 2× buffer (pH 7.5).

Wide field fluorescence imaging was conducted in an Olympus IX83 inverted microscope equipped with appropriate cubes for the dyes used (Table 4, Chroma) and 60× oil immersion objective (NA=1.4, Olympus). Images were captured with ORCA-R2 CCD camera (Hamamatsu) at intervals of 0.2 μm on the z-axis.

Following imaging, raw image stacks were processed in ImageJ (Schindelin J, Arganda-Carreras I, Frise E, Kaynig V, Longair M, et al. (2012) Fiji: an open-source platform for biological-image analysis. Nature methods 9: 676-682). First, the Laplacian of Gaussian filter was applied (Sage D, Neumann F R, Hediger F, Gasser S M, Unser M (2005) Automatic tracking of individual fluorescence particles: application to the study of chromosome dynamics. IEEE transactions on image processing: a publication of the IEEE Signal Processing Society 14: 1372-1383), considering the entire z-stack. Images where then inverted for black and white values, followed by reassignment of 16bit values to the gray depth. Second, the 3D rendered signal spots were counted (Bolte S, Cordelières F P (2006) A guided tour into subcellular colocalization analysis in light microscopy. Journal of microscopy 224: 213-232) at incrementing threshold intervals of 500 fluorescence arbitrary units (FIG. 7A to 7B). In addition, spots were considered representative of RNA molecules if their volume (voxel) was in the range of 8-125 pixel³. The number of spots in each image stack was obtained at the threshold interval of three consecutive equal counts (FIG. 7A to 7B). It was observed that selecting fluorescence intensities beyond this plateau one would have reduced the number of spots identified in the order of one unit, with the potential increase in the chance of not identifying a real spot. This criteria allowed quantification of spots reliably irrespective of the image background.

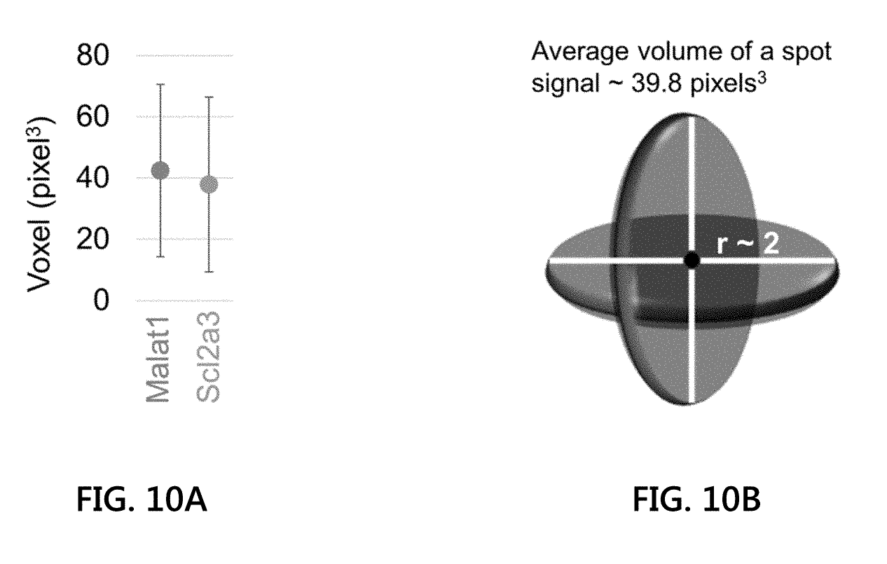

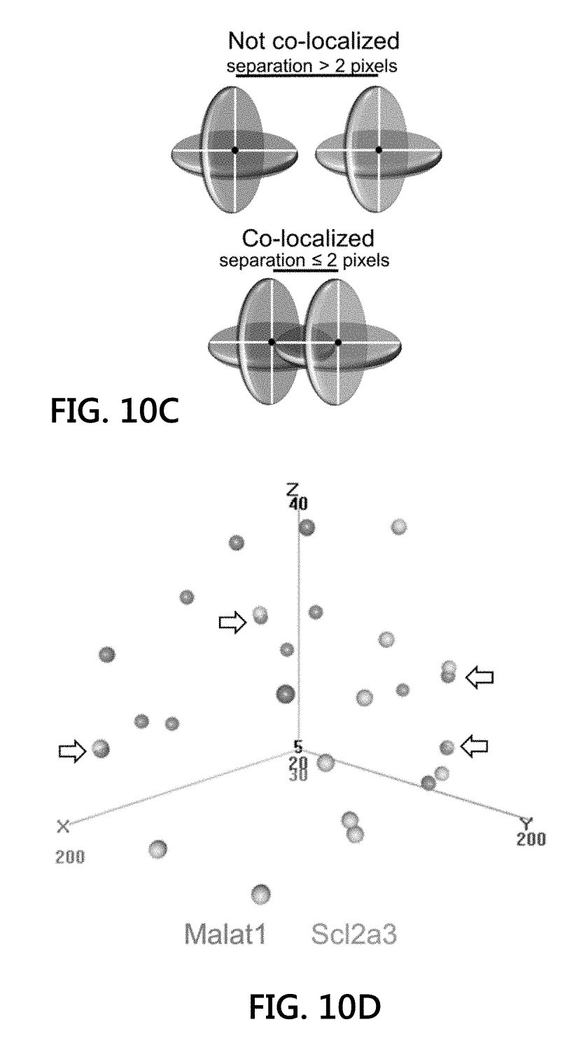

In addition to the counts, data was collected for the spot's respective x-y-z center of mass. Co-localization of two RNA molecules was inferred if the center of mass for two distinct spots, labeled with different dyes, were closer than 2 pixels (214 nm) in each of x, y, and z coordinates (FIG. 10A to 10D).

The following rationales to guide the optimization of the protocol for hybridization and imaging were used.

### Length and Number of the Oligonucleotides

Previous protocols for smRNA-FISH have used ≧12 oligonucleotides 20 bases long targeting the gene of interest. Here, 5 oligonucleotides 25-30 bases long were used. The signal detection of a spot corresponding to one mRNA molecule relies on the cumulative fluorescence intensity of each dye bound to the oligonucleotide probing the RNA molecule. Because QDs have greater intensity of fluorescence compared to organic dyes (Resch-Genger et al., 2008) the number of probes was reduced considerably. Working with 5 oligonucleotides 25-30 bases long allowed a fluorescent spot from background to be discerned, which is usually comprised of scattered single QDs. Previous research have shown that five oligonucleotides 30 bases long would achieve appropriate balance between sensitivity and specificity when quantifying relative abundance of a RNA molecule (Relogio et al., 2002).

### Finding the Appropriate Ratio of QDs and Oligonucleotides

The experiment to find appropriate ratio of quantum dots and oligonucleotides was performed to maximize the labeling of oligonucleotides with QDs. That also reduces the number or unattached oligonucleotides and QDs. Unlabeled oligonucleotides may compete for probe sites and if attached to the RNA molecule do not emit fluorescence. QDs not conjugated with oligonucleotides may not be washed away and will contribute to increased background.

### Probe Concentration of 10-50 μM

The current turbo fish protocol hybridizes probes at the concentration of 400 μM in for a period ranging from 30 seconds to 10 minutes. This current protocol works with concentration of 10-50 μM for each nucleotide. The probes used are 10 fold less concentrated to optimize our cost of per experiment. To compensate this the period of hybridization can be extended.

### Hybridization Period

As there are less probes in the hybridization buffer, the hybridization time was extended from 30 seconds to 30 minutes. In addition, QDs are larger than organic dyes (Resch-Genger et al., 2008) and may require longer time for the structure to penetrate the cells.

### Washing Periods

The probes were washed the for 30 minutes because the QDs are larger than organic dyes and may take longer to exit the cellular structures.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

### Additional Embodiments

In some embodiments, a method for detecting at least one target nucleic acid is provided, wherein the method comprises contacting said at least one target nucleic acid with a plurality of different nucleic acid probes which are associated with at least one detectable component with a high wavelength emission under conditions in which said plurality of different nucleic acid probes bind to said at least one target nucleic acid. In some embodiments, at least one detectable component comprises a particle with a high wavelength emission. In some embodiments, the plurality of different nucleic acid probes comprises at least one probe for at least one target nucleic acid. In some embodiments, a plurality of sets of probes with each of the sets of probes being associated with detection components which emit at different and distinguishable high wavelengths can be used to detect a plurality of target nucleic acids. In some embodiments, the nucleic acid probes are directed towards distinct sequences that are complimentary to the probes on one target nucleic acid strand. In some embodiments, the different probes target different or distinct target nucleic acid sequences on one target nucleic acid strand. In some embodiments, the plurality of different nucleic acid probes comprises at least two probes for at least one or two target nucleic acids. In some embodiments, the plurality of different nucleic acid probes comprises at least three probes for at least one, two or three target nucleic acids. In some embodiments, the plurality of different nucleic acid probes comprises at least four probes for at least one, two, three or four target nucleic acids. In some embodiments, the plurality of different nucleic acid probes comprises at least five probes for at least one, two, three, four or five target nucleic acids. In some embodiments, the plurality of different nucleic acid probes comprises at least six probes for at least one, two, three, four, five or six target nucleic acids. In some embodiments, the plurality of different nucleic acid probes comprises at least seven probes for at least one, two, three, four, five, six or seven target nucleic acids. In some embodiments, the plurality of different nucleic acid probes comprises at least eight probes for at least one, two, three, four, five, six, seven or eight target nucleic acids. In some embodiments, the plurality of different nucleic acid probes comprises at least nine probes for at least one, two, three, four, five, six, seven, eight or nine target nucleic acids. In some embodiments, the plurality of different nucleic acid probes comprises at least ten probes for at least one, two, three, four, five, six, seven, eight, nine or ten target nucleic acids. In some embodiments, the plurality of different nucleic acid probes comprise 5 or more different probes. In some embodiments, each of said plurality of different probes are between about 10, about 20, about 30, about 40, about 50, about 60, about 70, about 80, about 90 or about 100 nucleotides in length, or any other length between two aforementioned values. In some embodiments the quantum dot emits at a high emission wavelength of about 450 nm, about 500 nm, about 550 nm, about 600 nm, about 650 nm, about 700 nm, about 750 nm, about 800 nm or about 850 nm, or any other high emission wavelength between any two aforementioned values. In some embodiments described herein, the quantum dot comprises a size from about 1, about 2, about 3, about 4, about 5, about 6, about 7, about 8, about 9, about 10 or about 11 nanometers or any other size in between any two aforementioned values. In some embodiments, at least one detectable component comprises a particle with a high wavelength emission. In some embodiments, said particle is a quantum dot. In some embodiments, said target nucleic acid comprises RNA. In some embodiments, said target nucleic acid comprises a single RNA molecule within a single cell. In some embodiments, said plurality of different probes comprise 5 or more different probes. In some embodiments, said plurality of different probes is between about 10 and about 100 nucleotides in length. In some embodiments, said plurality of different probes are between about 10, about 20, about 30, about 40, about 50, about 60, about 70, about 80, about 90 or about 100 nucleotides in length, or any other length between two aforementioned values. In some embodiments, said plurality of different probes is between about 20 and about 80 nucleotides in length. In some embodiments, said plurality of different probes is about 30 nucleotides in length. In some embodiments, a plurality of target nucleic acids are detected. In some embodiments, the nucleic acid probe further comprises a nucleic acid linker, wherein the linker is not complementary to the target binding site, wherein the nucleic acid linker comprises about 3, about 4, about 5, about 6, about 7, about 8, about 9 or about 10 nucleic acids or any number of nucleic acids in between any two of the aforementioned values, and wherein the nucleic acid linker is covalently bound to the quantum dot. In some embodiments, wherein the contacting the probe to the target nucleic acid is performed for about 0.5 min, about 1 minute, about 5 minutes, about 10 minutes, about 20 minutes, about 30 minutes, or about 40 minutes, or any other time in between any two aforementioned values. In some embodiments, the method further comprises washing away the probes for 30 minutes after hybridization or contacting said at least one target nucleic acid. In some embodiments, the probes are at a concentration of about 10, about 20, about 30, about 40 or about 50 μM or any other concentration between any two aforementioned values. In some embodiments, the method further comprises single molecule analysis of RNA splicing and RNA-RNA interaction in vivo.

In some embodiments, a nucleic acid probe is provided. The nucleic acid probe can be associated with at least one detectable component with a high wavelength emission. In some embodiments, the at least one detectable component comprises a particle with a high wavelength emission. In some embodiments, said particle is a quantum dot. In some embodiments, the particle emits at a high emission wavelength of about 450 nm, about 500 nm, about 550 nm, about 600 nm, about 650 nm, about 700 nm, about 750 nm, about 800 nm or about 850 nm, or any other high emission wavelength between any two aforementioned values. In some embodiments, the particle or quantum dot comprises a size from about 1, about 2, about 3, about 4, about 5, about 6, about 7, about 8, about 9, about 10 or about 11 nanometers or any other size in between any two aforementioned values. In some embodiments, said at least one detectable component is covalently linked to said nucleic acid probe. In some embodiments, quantum dots can be conjugated to a nucleic acid probe in the presence of EDC an N-hydroxysuccinimide. In some embodiments, the quantum dot is conjugated to the nucleic acid by amine modification of the nucleic acid for coupling to quantum dots surfaces via the formation of an amide linkage. In some embodiments, the quantum dot is conjugated to the probe by biotinylation of the nucleic acid for attachment to quantum dots coated in streptavidin. In some embodiments, wherein the nucleic acid probe is complimentary to the target nucleic acid sequence, the probe further comprises a linker nucleic acid to which the QD is covalently attached. In some embodiments, the linker DNA allows freedom in the motion of the quantum dots during annealing and furthermore, can allow the quantum dot a distance from the annealing site in the event that the size of the QD can prevent annealing. In some embodiments, the nucleic acid probe is between about 10 and about 100 nucleotides in length. In some embodiments, the nucleic acid probe is about 10, about 20, about 30, about 40, about 50, about 60, about 70, about 80, about 90 or about 100 nucleotides long or any other length in between any two of the aforementioned values. In some embodiments, said nucleic acid probe is between about 20 and about 80 nucleotides in length. In some embodiments, the said nucleic acid probe is about 30 nucleotides in length.

In some embodiments, a kit is provided. The kit can comprise a plurality of different nucleic acid probes which are able to hybridize to at least one target nucleic acid, wherein each of said plurality of different nucleic acid probes is associated with at least one detectable component with a high wavelength emission. In some embodiments, said at least one detectable component comprises a particle with a high wavelength emission. In some embodiments, the particle emits at a high emission wavelength of about 450 nm, about 500 nm, about 550 nm, about 600 nm, about 650 nm, about 700 nm, about 750 nm, about 800 nm or about 850 nm, or any other high emission wavelength between any two aforementioned values. In some embodiments, the particle comprises a size from about 1, about 2, about 3, about 4, about 5, about 6, about 7, about 8, about 9, about 10 or about 11 nanometers or any other size in between any two aforementioned values. In some embodiments, said particle is a quantum dot. In some embodiments, said at least one detectable component is covalently linked to each of said plurality of different nucleic acid probes. In some embodiments, quantum dots can be conjugated to a nucleic acid probe in the presence of EDC an N-hydroxysuccinimide. In some embodiments, the quantum dot is conjugated to the nucleic acid by amine modification of the nucleic acid for coupling to quantum dots surfaces via the formation of an amide linkage. In some embodiments, the quantum dot is conjugated to the probe by biotinylation of the nucleic acid for attachment to quantum dots coated in streptavidin. In some embodiments, wherein the nucleic acid probe is complimentary to the target nucleic acid sequence, the probe further comprises a linker nucleic acid to which the QD is covalently attached.

In some embodiments, a method for detecting a plurality of target nucleic acids is provided, wherein the method comprises contacting said plurality of target nucleic acids with a plurality of sets of nucleic acid probes, wherein each set of nucleic acid probes comprises a plurality of different nucleic acid probes which are associated with at least one detectable component with a high wavelength emission, wherein each set of nucleic acid probes hybridizes to a different target nucleic acid, and wherein each set of nucleic acid probes is associated with a detectable component which emits at a high wavelength which is distinguishable from the high wavelength emissions of the detectable components associated with the other sets of nucleic acid probes and wherein said contacting is performed under conditions in which said plurality of sets of nucleic acid probes bind to said plurality of target nucleic acids. In some embodiments, said at least one detectable component comprises a particle with a high wavelength emission. In some embodiments, said particle is a quantum dot. In some embodiments, said target nucleic acid comprises Rain some embodiments, said target nucleic acid comprises DNA. In some embodiments, said target nucleic acid comprises a single RNA molecule within a single cell. In some embodiments, said target nucleic acid comprises a single DNA molecule within a single cell. In some embodiments, said plurality of different probes comprise 5 or more different probes. In some embodiments, said plurality of different probes is between about 10 and about 100 nucleotides in length. In some embodiments, said plurality of different probes is between about 20 and about 80 nucleotides in length. In some embodiments, each of said plurality of different probes is about 30 nucleotides in length. In some embodiments, a plurality of target nucleic acids are detected.

Each of the following references listed herein is incorporated herein by reference in its entirety.

### REFERENCES

1. Medintz I L, Uyeda H T, Goldman E R, Mattoussi H (2005) Quantum dot bioconjugates for imaging, labelling and sensing. Nat Mater 4: 435-446.
2. Valleron W, Laprevotte E, Gautier E F, Quelen C, Demur C, et al. (2012) Specific small nucleolar RNA expression profiles in acute leukemia. Leukemia 26: 2052-2060.
3. Schena M, Shalon D, Davis R W, Brown P O (1995) Quantitative monitoring of gene expression patterns with a complementary DNA microarray. Science 270: 467-470.
4. Chu Y, Corey D R (2012) RNA sequencing: platform selection, experimental design, and data interpretation. Nucleic Acid Ther 22: 271-274.
5. Nanostring (2014) nCounter Single Cell Gene Expression.
6. Ramskold D, Luo S, Wang Y C, Li R, Deng Q, et al. (2012) Full-length mRNA-Seq from single-cell levels of RNA and individual circulating tumor cells. Nat Biotechnol 30: 777-782.
7. Moignard V, Macaulay I C, Swiers G, Buettner F, Schutte J, et al. (2013) Characterization of transcriptional networks in blood stem and progenitor cells using high-throughput single-cell gene expression analysis. Nat Cell Biol 15: 363-372.
8. Raj A, Peskin C S, Tranchina D, Vargas D Y, Tyagi S (2006) Stochastic mRNA synthesis in mammalian cells. PLoS biology 4: e309.
9. Batish M, Raj A, Tyagi S (2011) Single molecule imaging of RNA in situ. Methods in molecular biology (Clifton, N J) 714: 3-13.
10. Raj A, van den Bogaard P, Rifkin S A, van Oudenaarden A, Tyagi S (2008) Imaging individual mRNA molecules using multiple singly labeled probes. Nature methods 5: 877-879.
11. Shaffer S M, Wu M-T, Levesque M J, Raj A (2013) Turbo FISH: A Method for Rapid Single Molecule RNA FISH. PloS one 8: e75120.
12. Lubeck E, Cai L (2012) Single-cell systems biology by super-resolution imaging and combinatorial labeling. Nat Methods 9: 743-748.
13. Takahashi H, Lassmann T, Murata M, Carninci P (2012) 5′ end-centered expression profiling using cap-analysis gene expression and next-generation sequencing. Nat Protoc 7: 542-561.
14. Ray D, Kazan H, Cook K B, Weirauch M T, Najafabadi H S, et al. (2013) A compendium of RNA-binding motifs for decoding gene regulation. Nature 499: 172-177.
15. Tripathi V, Ellis J D, Shen Z, Song D Y, Pan Q, et al. (2010) The nuclear-retained noncoding RNA MALAT1 regulates alternative splicing by modulating SR splicing factor phosphorylation. Mol Cell 39: 925-938.
16. Lichtman J W, Conchello J A (2005) Fluorescence microscopy. Nat Methods 2: 910-919.
17. Panchuk-Voloshina N, Haugland R P, Bishop-Stewart J, Bhalgat M K, Millard P J, et al. (1999) Alexa dyes, a series of new fluorescent dyes that yield exceptionally bright, photostable conjugates. J Histochem Cytochem 47: 1179-1188.
18. Resch-Genger U, Grabolle M, Cavaliere-Jaricot S, Nitschke R, Nann T (2008) Quantum dots versus organic dyes as fluorescent labels. Nature methods 5: 763-775.
19. Zrazhevskiy P, Gao X (2013) Quantum dot imaging platform for single-cell molecular profiling. Nat Commun 4: 1619.
20. Smith A M, Nie S (2012) Compact quantum dots for single-molecule imaging. J Vis Exp.
21. Roch N, Florens S, Bouchiat V, Wernsdorfer W, Balestro F (2008) Quantum phase transition in a single-molecule quantum dot. Nature 453: 633-637.
22. Chi S W, Zang J B, Mele A, Darnell R B (2009) Argonaute HITS-CLIP decodes microRNA-mRNA interaction maps. Nature 460: 479-486.
23. Helwak A, Kudla G, Dudnakova T, Tollervey D (2013) Mapping the human miRNA interactome by CLASH reveals frequent noncanonical binding. Cell 153: 654-665.
24. Batista P J, Chang H Y (2013) Long noncoding RNAs: cellular address codes in development and disease. Cell 152: 1298-1307.
25. Amaral P P, Dinger M E, Mercer T R, Mattick J S (2008) The eukaryotic genome as an RNA machine. Science 319: 1787-1789.
26. Kato Y, Sato K, Hamada M, Watanabe Y, Asai K, et al. (2010) RactIP: fast and accurate prediction of RNA-RNA interaction using integer programming. Bioinformatics 26: i460-466.
27. Wenzel A, Akbasli E, Gorodkin J (2012) RIsearch: fast RNA-RNA interaction search using a simplified nearest-neighbor energy model. Bioinformatics 28: 2738-2746.
28. Schneider C A, Rasband W S, Eliceiri K W (2012) NIH Image to ImageJ: 25 years of image analysis. Nature methods 9: 671-675.
29. Sage D, Neumann F R, Hediger F, Gasser S M, Unser M (2005) Automatic tracking of individual fluorescence particles: application to the study of chromosome dynamics. IEEE transactions on image processing: a publication of the IEEE Signal Processing Society 14: 1372-1383.
30. Bolte S, Cordelières F P (2006) A guided tour into subcellular colocalization analysis in light microscopy. Journal of microscopy 224: 213-232.
31. Hutchinson J N, Ensminger A W, Clemson C M, Lynch C R, Lawrence J B, et al. (2007) A screen for nuclear transcripts identifies two linked noncoding RNAs associated with SC35 splicing domains. BMC genomics 8: 39.
32. Ghavi-Helm Y, Klein F A, Pakozdi T, Ciglar L, Noordermeer D, et al. (2014) Enhancer loops appear stable during development and are associated with paused polymerase. Nature 512: 96-100.
33. Durisic N, Wiseman P W, Grater P, Heyes C D (2009) A common mechanism underlies the dark fraction formation and fluorescence blinking of quantum dots. ACS Nano 3: 1167-1175.
34. Yao J, Larson D R, Vishwasrao H D, Zipfel W R, Webb W W (2005) Blinking and nonradiant dark fraction of water-soluble quantum dots in aqueous solution. Proc Natl Acad Sci USA 102: 14284-14289.
35. Frantsuzov P A, Volkan-Kacso S, Janko B (2013) Universality of the fluorescence intermittency in nanoscale systems: experiment and theory. Nano Lett 13: 402-408.
36. Lee L Y, Ong S L, Hu J Y, Ng W J, Feng Y, et al. (2004) Use of semiconductor quantum dots for photostable immunofluorescence labeling of Cryptosporidium parvum. Appl Environ Microbiol 70: 5732-5736.
37. Jaiswal J K, Mattoussi H, Mauro J M, Simon S M (2003) Long-term multiple color imaging of live cells using quantum dot bioconjugates. Nature biotechnology 21: 47-51.
38. Wu X, Liu H, Liu J, Haley K N, Treadway J A, et al. (2003) Immunofluorescent labeling of cancer marker Her2 and other cellular targets with semiconductor quantum dots. Nature biotechnology 21: 41-46.
39. Han M, Gao X, Su J Z, Nie S (2001) Quantum-dot-tagged microbeads for multiplexed optical coding of biomolecules. Nature biotechnology 19: 631-635.
40. Goldman E R, Clapp A R, Anderson G P, Uyeda H T, Mauro J M, et al. (2004) Multiplexed toxin analysis using four colors of quantum dot fluororeagents. Anal Chem 76: 684-688.
41. Schindelin J, Arganda-Carreras I, Frise E, Kaynig V, Longair M, et al. (2012) Fiji: an open-source platform for biological-image analysis. Nature methods 9: 676-682.
42. Relogio, Biase F., Huang N., and Zhong S., (2012) Quantum dot based single molecule RNA-FISH for counting RNA molecules and visualizing RNA interactions. (Supplementary text)
43. Raj, A., Peskin, C. S., Tranchina, D., Vargas, D. Y., and Tyagi, S. (2006). Stochastic mRNA synthesis in mammalian cells. PLoS Biol. 4, e309.

44. Raj, A., van den Bogaard, P., Rifkin, S. A., van Oudenaarden, A., and Tyagi, S. (2008). Imaging individual mRNA molecules using multiple singly labeled probes. Nat. Methods 5, 877-879.
45. Batish, M., Raj, A., and Tyagi, S. (2011). Single molecule imaging of RNA in situ. Methods Mol. Biol. 714, 3-13. Raj, A., and Tyagi, S. (2010). Detection of individual endogenous RNA transcripts in situ using multiple singly labeled probes. Methods Enzymol. 472, 365-386.
46. Shaffer, S. M., Wu, M.-T., Levesque, M. J., and Raj, A. (2013). Turbo FISH: A Method for Rapid Single Molecule RNA FISH. PLoS One 8, e75120.
47. Yang, H., Wanner, I. B., Roper, S. D., and Chaudhari, N. (1999). An optimized method for in situ hybridization with signal amplification that allows the detection of rare mRNAs. J. Histochem. Cytochem. 47, 431-446.
48. Chan, P., Yuen, T., Ruf, F., Gonzalez-maeso, J., & Sealfon, S. C. (2005). Method for multiplex cellular detection of mRNAs using quantum dot fluorescent in situ hybridization. Nucleic acids research, 33(18), 1-8. doi:10.1093/nar/gni162
49. Choi, Y., Kim, H. P., Hong, S. M., Ryu, J. Y., Han, S. J., & Song, R. (2009). In situ Visualization of Gene Expression Using Polymer-Coated Quantum-Dot—DNA Conjugates. Small, 5(18), 2085-2091. doi:10.1002/sm11.200900116
50. Akita, H., Umetsu, Y., Kurihara, D., & Harashima, H. (2011). Dual imaging of mRNA and protein production: An investigation of the mechanism of heterogeneity in cationic lipid-mediated transgene expression. International Journal of Pharmaceutics, 415, 218-220. doi:10.1016/j.ijpharm.2011.05.051
51. Ioannou, A., Eleftheriou, I., Lubatti, A., Charalambous, A., & Skourides, P. A. (2012). High-resolution whole-mount in situ hybridization using Quantum Dot nanocrystals. Journal of biomedicine & biotechnology, 2012, 627602. doi:10.1155/2012/627602
52. Wu, S., Zhao, X., Zhang, Z., and Xie, H. (2006). Quantum-Dot-Labeled DNA Probes for Fluorescence In Situ Hybridization (FISH) in the Microorganism Escherichia coli. 1062-1067.
53. Smith, A. M., Duan, H., Mohs, A. M., and Nie, S. (2008). Bioconjugated quantum dots for in vivo molecular and cellular imaging. Adv. Drug Deliv. Rev. 60, 1226-1240.
54. Friedrich, M., Nozadze, R., Gan, Q., Zelman-femiak, M., Ermolayev, V., Wagner, T. U., and Harms, G. S. (2009). Biochemical and Biophysical Research Communications Detection of single quantum dots in model organisms with sheet illumination microscopy. Biochem. Biophys. Res. Commun. 390, 722-727.
55. Yang, H., Wanner, I. B., Roper, S. D., and Chaudhari, N. (1999). An optimized method for in situ hybridization with signal amplification that allows the detection of rare mRNAs. J. Histochem. Cytochem. 47, 431-446.
56. Chan, P., Yuen, T., Ruf, F., Gonzalez-maeso, J., & Sealfon, S. C. (2005). Method for multiplex cellular detection of mRNAs using quantum dot fluorescent in situ hybridization. Nucleic acids research, 33(18), 1-8. doi:10.1093/nar/gni162
57. Choi, Y., Kim, H. P., Hong, S. M., Ryu, J. Y., Han, S. J., & Song, R. (2009). In situ Visualization of Gene Expression Using Polymer-Coated Quantum-Dot—DNA Conjugates. Small, 5(18), 2085-2091. doi:10.1002/sm11.200900116
58. Akita, H., Umetsu, Y., Kurihara, D., & Harashima, H. (2011). Dual imaging of mRNA and protein production: An investigation of the mechanism of heterogeneity in cationic lipid-mediated transgene expression. International Journal of Pharmaceutics, 415, 218-220. doi:10.1016/j.ijpharm.2011.05.051
59. Ioannou, A., Eleftheriou, I., Lubatti, A., Charalambous, A., & Skourides, P. A. (2012). High-resolution whole-mount in situ hybridization using Quantum Dot nanocrystals. Journal of biomedicine & biotechnology, 2012, 627602. doi:10.1155/2012/627602
60. Raj, A., van den Bogaard, P., Rifkin, S. A., van Oudenaarden, A., & Tyagi, S. (2008). Imaging individual mRNA molecules using multiple singly labeled probes. Nature methods, 5(10), 877-9. doi:10.1038/nmeth.1253
61. Batish, M., Raj, A., & Tyagi, S. (2011). Single molecule imaging of RNA in situ. Methods in molecular biology (Clifton, N. J.), 714, 3-13. doi:10.1007/978-1-61779-005-8_1
62. Raj, A., & Tyagi, S. (2010). Detection of individual endogenous RNA transcripts in situ using multiple singly labeled probes. Methods in enzymology, 472, 365-86. doi:10.1016/S0076-6879(10)72004-8
63. Shaffer, S. M., Wu, M.-T., Levesque, M. J., & Raj, A. (2013). Turbo FISH: A Method for Rapid Single Molecule RNA FISH. PloS one, 8(9), e75120. doi:10.1371/journal.pone.0075120
64. Yang, H., Wanner, I. B., Roper, S. D., and Chaudhari, N. (1999). An optimized method for in situ hybridization with signal amplification that allows the detection of rare mRNAs. J. Histochem. Cytochem. 47, 431-446.
65. Chan, P., Yuen, T., Ruf, F., Gonzalez-maeso, J., & Sealfon, S. C. (2005). Method for multiplex cellular detection of mRNAs using quantum dot fluorescent in situ hybridization. Nucleic acids research, 33(18), 1-8. doi:10.1093/nar/gni162
66. Choi, Y., Kim, H. P., Hong, S. M., Ryu, J. Y., Han, S. J., & Song, R. (2009). In situ Visualization of Gene Expression Using Polymer-Coated Quantum-Dot—DNA Conjugates. Small, 5(18), 2085-2091. doi:10.1002/sm11.200900116
67. Akita, H., Umetsu, Y., Kurihara, D., & Harashima, H. (2011). Dual imaging of mRNA and protein production: An investigation of the mechanism of heterogeneity in cationic lipid-mediated transgene expression. International Journal of Pharmaceutics, 415, 218-220. doi:10.1016/j.ijpharm.2011.05.051
68. Ioannou, A., Eleftheriou, I., Lubatti, A., Charalambous, A., & Skourides, P. A. (2012). High-resolution whole-mount in situ hybridization using Quantum Dot nanocrystals. Journal of biomedicine & biotechnology, 2012, 627602. doi:10.1155/2012/627602
69. Raj, A., van den Bogaard, P., Rifkin, S. A., van Oudenaarden, A., & Tyagi, S. (2008). Imaging individual mRNA molecules using multiple singly labeled probes. Nature methods, 5(10), 877-9. doi:10.1038/nmeth.1253
70. Batish, M., Raj, A., & Tyagi, S. (2011). Single molecule imaging of RNA in situ. Methods in molecular biology (Clifton, N. J.), 714, 3-13. doi:10.1007/978-1-61779-005-8_1
71. Raj, A., & Tyagi, S. (2010). Detection of individual endogenous RNA transcripts in situ using multiple singly labeled probes. Methods in enzymology, 472, 365-86. doi:10.1016/S0076-6879(10)72004-8
72. Shaffer, S. M., Wu, M.-T., Levesque, M. J., & Raj, A. (2013). Turbo FISH: A Method for Rapid Single Molecule RNA FISH. PloS one, 8(9), e75120. doi:10.1371/journal.pone.0075120

1. A method for detecting at least one target nucleic acid comprising contacting said at least one target nucleic acid with a plurality of different nucleic acid probes which are associated with at least one detectable component with a high wavelength emission under conditions in which said plurality of different nucleic acid probes bind to said at least one target nucleic acid.

2. The method of claim 1, wherein said at least one detectable component comprises a particle with a high wavelength emission.

3. The method of claim 2, wherein said particle is a quantum dot.

4. The method of claim 1, wherein said target nucleic acid comprises RNA.

5. The method of claim 1, wherein said target nucleic acid comprises a single RNA molecule within a single cell.

6. The method of claim 1, wherein said plurality of different probes comprise 5 or more different probes.

7. The method of claim 1, wherein each of said plurality of different probes is between about 10 and about 100 nucleotides in length.

8. The method of claim 1, wherein each of said plurality of different probes is between about 20 and about 80 nucleotides in length.

9. The method of claim 1, wherein each of said plurality of different probes is about 30 nucleotides in length.

10. The method of claim 1, wherein a plurality of target nucleic acids are detected.

11. A nucleic acid probe associated with at least one detectable component with a high wavelength emission.

12. The nucleic acid probe of claim 11, wherein said at least one detectable component comprises a particle with a high wavelength emission.

13. The nucleic acid probe of claim 12, wherein said particle is a quantum dot.

14. The nucleic acid probe of claim 11, wherein said at least one detectable component is covalently linked to said nucleic acid probe.

15. The nucleic acid probe of claim 11, wherein said nucleic acid probe is between about 10 and about 100 nucleotides in length.

16. The nucleic acid probe of claim 11, wherein said nucleic acid probe is between about 20 and about 80 nucleotides in length.

17. The nucleic acid probe of claima 11, wherein said nucleic acid probe is about 30 nucleotides in length.

18. A kit comprising a plurality of different nucleic acid probes which are able to hybridize to at least one target nucleic acid, wherein each of said plurality of different nucleic acid probes is associated with at least one detectable component with a high wavelength emission.

19. The kit of claim 18, wherein said at least one detectable component comprises a particle with a high wavelength emission.

20. The kit of any one of claim 19, wherein said particle is a quantum dot.

21. The kit of claim 18, wherein said at least one detectable component is covalently linked to each of said plurality of different nucleic acid probes.

22. A method for detecting a plurality of target nucleic acids comprising contacting said plurality of target nucleic acids with a plurality of sets of nucleic acid probes, wherein each set of nucleic acid probes comprises a plurality of different nucleic acid probes which are associated with at least one detectable component with a high wavelength emission, wherein each set of nucleic acid probes hybridizes to a different target nucleic acid, and wherein each set of nucleic acid probes is associated with a detectable component which emits at a high wavelength which is distinguishable from the high wavelength emissions of the detectable components associated with the other sets of nucleic acid probes and wherein said contacting is performed under conditions in which said plurality of sets of nucleic acid probes bind to said plurality of target nucleic acids.


SEQUENCE LISTING (60 sequences)

SEQ ID NO: 1 - DNA, 30 bp, Artificial Sequence
  Synthetic oligonucleotide
tctcaaacatgatctgggtcatcttttcac

SEQ ID NO: 2 - DNA, 30 bp, Artificial Sequence
  Synthetic oligonucleotide
gttggcatagaggtctttacggatgtcaac

SEQ ID NO: 3 - DNA, 30 bp, Artificial Sequence
  Synthetic oligonucleotide
caatgatcttgatcttcatggtgctaggag

SEQ ID NO: 4 - DNA, 30 bp, Artificial Sequence
  Synthetic oligonucleotide
ttttgtcaaagaaagggtgtaaaacgcagc

SEQ ID NO: 5 - DNA, 30 bp, Artificial Sequence
  Synthetic oligonucleotide
cgttccagtttttaaatcctgagtcaaaag

SEQ ID NO: 6 - DNA, 24 bp, Artificial Sequence
  Synthetic oligonucleotide
aaggtctcacatcacacactcact

SEQ ID NO: 7 - DNA, 24 bp, Artificial Sequence
  Synthetic oligonucleotide
caaagaacagacatgacctgaagt

SEQ ID NO: 8 - DNA, 25 bp, Artificial Sequence
  Synthetic oligonucleotide
ttctaatagcagcagattggaacag

SEQ ID NO: 9 - DNA, 24 bp, Artificial Sequence
  Synthetic oligonucleotide
aacaaccactactccaaacacttg

SEQ ID NO: 10 - DNA, 24 bp, Artificial Sequence
  Synthetic oligonucleotide
ctcaacactcagcctgttactcat

SEQ ID NO: 11 - DNA, 25 bp, Artificial Sequence
  Synthetic oligonucleotide
tagccacaatacagacaaagctcat

SEQ ID NO: 12 - DNA, 25 bp, Artificial Sequence
  Synthetic oligonucleotide
ctcaaagaaggctacgtagatcaag

SEQ ID NO: 13 - DNA, 24 bp, Artificial Sequence
  Synthetic oligonucleotide
agaaagttggaggtccagttacaa

SEQ ID NO: 14 - DNA, 25 bp, Artificial Sequence
  Synthetic oligonucleotide
cgtccttgaagattcctgttgagta

SEQ ID NO: 15 - DNA, 23 bp, Artificial Sequence
  Synthetic oligonucleotide
cctatcatatgcagggttctcct

SEQ ID NO: 16 - DNA, 20 bp, Artificial Sequence
  Synthetic oligonucleotide
tgcaaagaagctgtgctcgc

SEQ ID NO: 17 - DNA, 20 bp, Artificial Sequence
  Synthetic oligonucleotide
tgtggaccggcaacgaagga

SEQ ID NO: 18 - DNA, 20 bp, Artificial Sequence
  Synthetic oligonucleotide
atatcgtcatccatggcgaa

SEQ ID NO: 19 - DNA, 20 bp, Artificial Sequence
  Synthetic oligonucleotide
acgatggaggggaatacagc

SEQ ID NO: 20 - DNA, 20 bp, Artificial Sequence
  Synthetic oligonucleotide
cacataggagtccttctgac

SEQ ID NO: 21 - DNA, 20 bp, Artificial Sequence
  Synthetic oligonucleotide
gtacttcagggtcaggatac

SEQ ID NO: 22 - DNA, 20 bp, Artificial Sequence
  Synthetic oligonucleotide
gttggtaacaatgccatgtt

SEQ ID NO: 23 - DNA, 20 bp, Artificial Sequence
  Synthetic oligonucleotide
acacgcagctcattgtagaa

SEQ ID NO: 24 - DNA, 20 bp, Artificial Sequence
  Synthetic oligonucleotide
tgatctgggtcatcttttca

SEQ ID NO: 25 - DNA, 20 bp, Artificial Sequence
  Synthetic oligonucleotide
ggggtgttgaaggtctcaaa

SEQ ID NO: 26 - DNA, 20 bp, Artificial Sequence
  Synthetic oligonucleotide
ctggatggctacgtacatgg

SEQ ID NO: 27 - DNA, 20 bp, Artificial Sequence
  Synthetic oligonucleotide
agaggcatacagggacagca

SEQ ID NO: 28 - DNA, 20 bp, Artificial Sequence
  Synthetic oligonucleotide
catcacaatgcctgtggtac

SEQ ID NO: 29 - DNA, 20 bp, Artificial Sequence
  Synthetic oligonucleotide
tcgtagatgggcacagtgtg

SEQ ID NO: 30 - DNA, 20 bp, Artificial Sequence
  Synthetic oligonucleotide
atggcgtgagggagagcata

SEQ ID NO: 31 - DNA, 20 bp, Artificial Sequence
  Synthetic oligonucleotide
atcttcatgaggtagtctgt

SEQ ID NO: 32 - DNA, 20 bp, Artificial Sequence
  Synthetic oligonucleotide
ctgtggtggtgaagctgtag

SEQ ID NO: 33 - DNA, 20 bp, Artificial Sequence
  Synthetic oligonucleotide
ttgatgtcacgcacgatttc

SEQ ID NO: 34 - DNA, 20 bp, Artificial Sequence
  Synthetic oligonucleotide
atctcctgctcgaagtctag

SEQ ID NO: 35 - DNA, 20 bp, Artificial Sequence
  Synthetic oligonucleotide
tagtttcatggatgccacag

SEQ ID NO: 36 - DNA, 20 bp, Artificial Sequence
  Synthetic oligonucleotide
ggtctttacggatgtcaacg

SEQ ID NO: 37 - DNA, 20 bp, Artificial Sequence
  Synthetic oligonucleotide
gacagcactgtgttggcata

SEQ ID NO: 38 - DNA, 20 bp, Artificial Sequence
  Synthetic oligonucleotide
tgggtacatggtggtaccac

SEQ ID NO: 39 - DNA, 20 bp, Artificial Sequence
  Synthetic oligonucleotide
agagcagtaatctccttctg

SEQ ID NO: 40 - DNA, 20 bp, Artificial Sequence
  Synthetic oligonucleotide
gatcttcatggtgctaggag

SEQ ID NO: 41 - DNA, 20 bp, Artificial Sequence
  Synthetic oligonucleotide
gctcaggaggagcaatgatc

SEQ ID NO: 42 - DNA, 20 bp, Artificial Sequence
  Synthetic oligonucleotide
ccgatccacacagagtactt

SEQ ID NO: 43 - DNA, 20 bp, Artificial Sequence
  Synthetic oligonucleotide
acagtgaggccaggatggag

SEQ ID NO: 44 - DNA, 20 bp, Artificial Sequence
  Synthetic oligonucleotide
gatccacatctgctggaagg

SEQ ID NO: 45 - DNA, 20 bp, Artificial Sequence
  Synthetic oligonucleotide
actcatcgtactcctgcttg

SEQ ID NO: 46 - DNA, 20 bp, Artificial Sequence
  Synthetic oligonucleotide
tagaagcacttgcggtgcac

SEQ ID NO: 47 - DNA, 20 bp, Artificial Sequence
  Synthetic oligonucleotide
aacgcagctcagtaacagtc

SEQ ID NO: 48 - DNA, 20 bp, Artificial Sequence
  Synthetic oligonucleotide
ggttttgtcaaagaaagggt

SEQ ID NO: 49 - DNA, 20 bp, Artificial Sequence
  Synthetic oligonucleotide
ttcaccgttccagtttttaa

SEQ ID NO: 50 - DNA, 20 bp, Artificial Sequence
  Synthetic oligonucleotide
atgtttgctccaaccaactg

SEQ ID NO: 51 - DNA, 20 bp, Artificial Sequence
  Synthetic oligonucleotide
tcagccacatttgtagaact

SEQ ID NO: 52 - DNA, 20 bp, Artificial Sequence
  Synthetic oligonucleotide
cctgtaaccacttatttcat

SEQ ID NO: 53 - DNA, 20 bp, Artificial Sequence
  Synthetic oligonucleotide
cttttgggagggtgagggac

SEQ ID NO: 54 - DNA, 20 bp, Artificial Sequence
  Synthetic oligonucleotide
cacagaagcaatgctgtcac

SEQ ID NO: 55 - DNA, 20 bp, Artificial Sequence
  Synthetic oligonucleotide
aaaaagggaggcctcagacc

SEQ ID NO: 56 - DNA, 20 bp, Artificial Sequence
  Synthetic oligonucleotide
gaccaaagccttcatacatc

SEQ ID NO: 57 - DNA, 20 bp, Artificial Sequence
  Synthetic oligonucleotide
ttggtctcaagtcagtgtac

SEQ ID NO: 58 - DNA, 19 bp, Artificial Sequence
  Synthetic oligonucleotide
gtgtaaggtaaggtgtgca

SEQ ID NO: 59 - RNA, 1889 nt, Mus musculus
  CDS (80)...(1207)
cugucgagucgcguccacccgcgagcacagcuucuuugcagcuccuucguugccgguccacacccgccaccaguucgccauggaugacgauaucgcugcgcuggucgucgacaacggcuccggcaugugcaaagccggcuucgcgggcgacgaugcuccccgggcuguauuccccuccaucgugggccgcccuaggcaccagggugugauggugggaaugggucagaaggacuccuaugugggugacgaggcccagagcaagagagguauccugacccugaaguaccccauugaacauggcauuguuaccaacugggacgacauggagaagaucuggcaccacaccuucuacaaugagcugcguguggccccugaggagcacccugugcugcucaccgaggccccccugaacccuaaggccaaccgugaaaagaugacccagaucauguuugagaccuucaacaccccagccauguacguagccauccaggcugugcugucccuguaugccucuggucguaccacaggcauugugauggacuccggagacggggucacccacacugugcccaucuacgagggcuaugcucucccucacgccauccugcgucuggaccuggcuggccgggaccugacagacuaccucaugaagauccugaccgagcguggcuacagcuucaccaccacagcugagagggaaaucgugcgugacaucaaagagaagcugugcuauguugcucuagacuucgagcaggagauggccacugccgcauccucuuccucccuggagaagagcuaugagcugccugacggccaggucaucacuauuggcaacgagcgguuccgaugcccugaggcucuuuuccagccuuccuucuuggguauggaauccuguggcauccaugaaacuacauucaauuccaucaugaagugugacguugacauccguaaagaccucuaugccaacacagugcugucuggugguaccaccauguacccaggcauugcugacaggaugcagaaggagauuacugcucuggcuccuagcaccaugaagaucaagaucauugcuccuccugagcgcaaguacucuguguggaucgguggcuccauccuggccucacuguccaccuuccagcagauguggaucagcaagcaggaguacgaugaguccggccccuccaucgugcaccgcaagugcuucuaggcggacuguuacugagcugcguuuuacacccuuucuuugacaaaaccuaacuugcgcagaaaaaaaaaaaauaagagacaacauuggcauggcuuuguuuuuuuaaauuuuuuuuaaaguuuuuuuuuuuuuuuuuuuuuuuuuuuuuaaguuuuuuuguuuuguuuuggcgcuuuugacucaggauuuaaaaacuggaacggugaaggcgacagcaguugguuggagcaaacaucccccaaaguucuacaaauguggcugaggacuuuguacauuguuuuguuuuuuuuuuuuuuugguuuugucuuuuuuuaauagucauuccaaguauccaugaaauaagugguuacaggaagucccucacccucccaaaagccacccccacuccuaagaggaggauggucgcguccaugcccugaguccaccccggggaaggugacagcauugcuucuguguaaauuauguacugcaaaaauuuuuuuaaaucuuccgccuuaauacuucauuuuuguuuuuaauuucugaauggcccaggucugaggccucccuuuuuuuuguccccccaacuugauguaugaaggcuuuggucucccugggaggggguugagguguugaggcagccagggcuggccuguacacugacuugagaccaauaaaagugcacaccuuaccuuacacaaac

SEQ ID NO: 60 - DNA, 1889 bp, Artificial Sequence
  CDS (80)...(1207)
  Beta actin cDNA
ctgtcgagtcgcgtccacccgcgagcacagcttctttgcagctccttcgttgccggtccacacccgccaccagttcgccatggatgacgatatcgctgcgctggtcgtcgacaacggctccggcatgtgcaaagccggcttcgcgggcgacgatgctccccgggctgtattcccctccatcgtgggccgccctaggcaccagggtgtgatggtgggaatgggtcagaaggactcctatgtgggtgacgaggcccagagcaagagaggtatcctgaccctgaagtaccccattgaacatggcattgttaccaactgggacgacatggagaagatctggcaccacaccttctacaatgagctgcgtgtggcccctgaggagcaccctgtgctgctcaccgaggcccccctgaaccctaaggccaaccgtgaaaagatgacccagatcatgtttgagaccttcaacaccccagccatgtacgtagccatccaggctgtgctgtccctgtatgcctctggtcgtaccacaggcattgtgatggactccggagacggggtcacccacactgtgcccatctacgagggctatgctctccctcacgccatcctgcgtctggacctggctggccgggacctgacagactacctcatgaagatcctgaccgagcgtggctacagcttcaccaccacagctgagagggaaatcgtgcgtgacatcaaagagaagctgtgctatgttgctctagacttcgagcaggagatggccactgccgcatcctcttcctccctggagaagagctatgagctgcctgacggccaggtcatcactattggcaacgagcggttccgatgccctgaggctcttttccagccttccttcttgggtatggaatcctgtggcatccatgaaactacattcaattccatcatgaagtgtgacgttgacatccgtaaagacctctatgccaacacagtgctgtctggtggtaccaccatgtacccaggcattgctgacaggatgcagaaggagattactgctctggctcctagcaccatgaagatcaagatcattgctcctcctgagcgcaagtactctgtgtggatcggtggctccatcctggcctcactgtccaccttccagcagatgtggatcagcaagcaggagtacgatgagtccggcccctccatcgtgcaccgcaagtgcttctaggcggactgttactgagctgcgttttacaccctttctttgacaaaacctaacttgcgcagaaaaaaaaaaaataagagacaacattggcatggctttgtttttttaaattttttttaaagtttttttttttttttttttttttttttttaagtttttttgttttgttttggcgcttttgactcaggatttaaaaactggaacggtgaaggcgacagcagttggttggagcaaacatcccccaaagttctacaaatgtggctgaggactttgtacattgttttgtttttttttttttttggttttgtctttttttaatagtcattccaagtatccatgaaataagtggttacaggaagtccctcaccctcccaaaagccacccccactcctaagaggaggatggtcgcgtccatgccctgagtccaccccggggaaggtgacagcattgcttctgtgtaaattatgtactgcaaaaatttttttaaatcttccgccttaatacttcatttttgtttttaatttctgaatggcccaggtctgaggcctcccttttttttgtccccccaacttgatgtatgaaggctttggtctccctgggagggggttgaggtgttgaggcagccagggctggcctgtacactgacttgagaccaataaaagtgcacaccttaccttacacaaac